(19) United States
(12) Patent Application Publication
(10) Pub. No.: US 2026/0117207 A1
(43) Pub. Date: Apr. 30, 2026

(54) PARENT PHYTASE VARIANT

(71) Applicant: Nanjing Bestzyme Bio-Engineering Co., Ltd., Nanjing (CN)

(72) Inventors: Jie Lin, Nanjing (CN); Aixi Bai, Nanjing (CN); Ke Huang, Nanjing (CN); Yan Sun, Nanjing (CN); Hong Xu, Nanjing (CN)

(21) Appl. No.: 19/436,528

(22) Filed: Dec. 30, 2025

Related U.S. Application Data
(62) Division of application No. 17/997,037, now Pat. No. 12,534,712.

(30) Foreign Application Priority Data
Apr. 29, 2020 (CN) 202010355432.X

(51) Int. Cl. C12N 9/16

(52) U.S. Cl. CPC C12N 9/16; C12Y 301/03026

(57) ABSTRACT

Provided is a parent phytase variant, which relates to the technical field of protein engineering. The variant, relative to the parent phytase thereof, has one or more amino acid substitutions at positions corresponding to positions 295, 349, and 374 of SEQ ID NO: 1. Compared to the parent phytase, the variant has increased thermal stability.

### CROSS-REFERENCE TO RELATED APPLICATIONS

The present application is a divisional application of U.S. Ser. No. 17/997,037, filed Oct. 25, 2022, which is a Section 371 of International Application No. PCT/CN2021/075123, filed on Feb. 3, 2021, which published in the Chinese language on Nov. 4, 2021, under International Publication No. WO 2021/218269 A1, and which claims priority to Chinese Patent Application No. CN 202010355432. X filed on Apr. 29, 2020. Each disclosure is incorporated herein by reference in its entirety.

### REFERENCE TO SEQUENCE LISTING SUBMITTED ELECTRONICALLY

This application contains a sequence listing, which is submitted electronically. The contents of the electronic sequence listing (file name: 065824.18US2 SequenceListing.xml; size: 194,027 bytes; creation date: Dec. 29, 2025) is herein incorporated by reference in its entirety.

### TECHNICAL FIELD

The present invention relates to the technical field of protein engineering. More specifically, the present invention relates to a parent phytase variant.

### BACKGROUND ART

Phytase, also known as myo-inositol hexaphosphate phosphohydrolase, is an orthophosphoric-monoester phosphohydrolase and is a general term for enzymes that catalyze the hydrolysis of phytic acid and phytate into inositol and phosphoric acid (phosphate). Phytic acid is most abundant in seeds of crops such as grains, beans, and oil plants, and is present in an amount of up to 1% to 3%, accounting for 60% to 80% of the total phosphorus content of plants. However, the phosphorus in phytic acid cannot be directly absorbed and used, and must be hydrolyzed into inorganic phosphate in the digestive tract first. Studies have shown that monogastric animals (pigs, chickens, ducks, geese, etc.) have a low utilization rate of phosphorus in phytic acid due to the lack of phytase. Furthermore, phytic acid, due to the strong electronegativity thereof, generally forms insoluble salts with divalent or trivalent cations, such as Ca²⁺, Zn²⁺ and Fe²⁺, which hinders the absorption of minerals in the small intestine. Phytic acid may also form complexes with proteins, amino acids, fatty acids, etc., thereby affecting the absorption and utilization thereof. Phytic acid may also bind to pepsin, chymotrypsin, trypsin, etc., which reduces the digestive enzyme activity. Therefore, adding phytase to feeds for monogastric animals can improve a utilization rate of phosphorus in animal feeds, reduce a phosphorus content in animal excrements and improve an energy utilization rate of proteins and feeds.

Phytase as a feed additive is pre-added to feed raw materials, the mixture is subject to high temperature granulation and other processes (at 70° C.-95° C. for 30-120 seconds), and then feeds are produced for animal feeding. The stress resistance, especially the thermal stability of phytase cannot meet requirements of feeds and feed processing, which has become one of the important limiting factors that restrict popularization and application of phytase. In view of the important significance of phytase in improving production efficiency of animal husbandry and reducing environmental pollution of phosphorus, improving thermal resistance of phytase has become a new research hotspot in the field of scientific research.

The use of coating technology can effectively reduce the activity loss of phytase during high temperature and high humidity treatment such as granulation, thereby greatly improving the utilization efficiency of phytase. Some progress has been made in the research of coating of phytase at home and abroad. The phytase coated by means of the phytase coating process developed by the famous enzyme preparation manufacturers Novozymes and BASF has greatly improved thermal stability compared with the uncoated phytase powder. Patents WO 2007044968, CN 1981597 A and CN 101168734 A all disclose the coating process for phytase. Although the coating process can significantly improve thermal resistance of phytase, the coating process is complicated, which may result in a prolonged production cycle of phytase and greatly increase the production cost of phytase.

In addition, on the basis of the amino acid sequence of wild-type phytase, phytase with excellent thermal resistance can also be obtained by mutating amino acids at specific positions by means of a protein engineering. Patents WO 2019228441 A1, U.S. Pat. No. 9,605,245, EP 2102334 B1, etc. all disclose subjecting specific amino acids of phytase to site-directed mutations, so that the finally obtained phytase products have improved thermal resistance compared to wild-type phytase. However, the enzymatic activity of these phytase products will still be largely lost during high temperature granulation of feeds.

Therefore, there is also a need to develop a phytase product with more excellent thermal resistance on the basis of the existing research.

### SUMMARY OF THE INVENTION

The object of the present invention is to provide a phytase product with more excellent thermal resistance by means of a protein engineering.

In order to achieve the above-mentioned object, the technical solution used in the present invention is as follows.

The inventors of the present invention discovered that, on the basis of a parent phytase, one or more amino acid substitutions are introduced at positions corresponding to positions 295, 349 and 374 of SEQ ID NO: 1, and the variant with phytase activity obtained thereby has significantly increased thermal stability compared to the parent phytase thereof, wherein the amino acid sequence of the parent phytase has at least 80% sequence identity to the amino acid sequence as shown in SEQ ID NO: 98.

In some preferred embodiments, the amino acid sequence of the parent phytase has at least 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence as shown in SEQ ID NO: 98; and further preferably, the amino acid sequence of the parent phytase is as shown in SEQ ID NO: 98.

In some specific embodiments, the amino acid sequence of the parent phytase is selected from SEQ ID NO: 2, SEQ ID NO: 98, SEQ ID NO: 102, SEQ ID NO: 107, SEQ ID NO: 112, or SEQ ID NO: 115.

In some embodiments, the amino acid at position 295 of the variant is substituted with P, Y, G, K, L or Q; preferably, P or Y; and further preferably, Y.

In some embodiments, the amino acid at position 349 of the variant is substituted with K, L, G, H, R, or T; preferably, K or L; and further preferably, K.

In some embodiments, the amino acid at position 374 of the variant is substituted with R, V, N, S, T, F, K, P or Y; preferably, R or V; and further preferably, R.

In some preferred embodiments, the variant, relative to the parent phytase thereof, comprises any amino acid substitutions selected from the group consisting of:

$$T 295 P ; Q 349 K ; E 3 \; 74 R ; T 295 Y ; Q 349 L ; E 3 \; 74 V ;$$

$$T 295 P + Q 349 K ; T 295 P + E 374 R ; T 295 P + Q 3 \; 49 L ;$$

$$T 295 P + E 374 V ; T 295 Y + Q 349 K ; T 295 Y + E 3 \; 74 R ;$$

$$T 295 Y + Q 349 L ; T 295 Y + E 374 V ; Q 349 L + E 3 \; 74 R ;$$

$$Q 349 K + E 374 R ; Q 349 L + E 374 V ; Q 349 K + E 3 \; 74 V ;$$

$$T 295 P + Q 349 K + E 3 \; 74 R ;$$

$$T 295 Y + Q 349 K + E 3 \; 74 R ;$$

$$T 295 P + Q 349 L + E 3 \; 74 R ;$$

$$T 295 Y + Q 349 L + E 3 \; 74 R ;$$

$$T 295 P + Q 349 K + E 3 \; 74 V ;$$

$$T 295 Y + Q 349 K + E 3 \; 74 V ;$$

$$T 295 P + Q 349 L + E 3 \; 74 V ;$$

$$T 295 Y + Q 349 L + E 374 V .$$

It should be noted that, the variant, relative to the parent phytase thereof, may only comprise amino acid substitutions selected from any of the above-mentioned groups, or may comprise some other mutation sites in addition to the amino acid substitutions selected from any of the above-mentioned groups, such as phytase mutation sites that have been disclosed in the prior art and can improve thermal resistance of phytase.

Preferably, the variant has at least 90%, 91%, 92%, 93%, 94%, 95%, 96%, 97%, 98% or 99%, but less than 100% sequence identity to the amino acid sequence of the parent phytase thereof.

In some embodiments, the variant further comprises at least one pair of introduced disulfide bonds, for example, any one or more pairs of disulfide bonds disclosed in patent WO 2019228441 A1; preferably, the variant comprises disulfide bonds formed between an amino acid residue at a position corresponding to position 346 of SEQ ID NO: 1 and an amino acid residue at a position corresponding to position 393 of SEQ ID NO: 1; and further preferably, the amino acid of the variant at a position corresponding to position 346 of SEQ ID NO: 1 is substituted with C, and the amino acid of the variant at a position corresponding to position 393 of SEQ ID NO: 1 is substituted with C.

It will be understood by a person skilled in the art that “introduction” herein does not define the formation of the disulfide bonds in any particular manner. For example, “introduction” of a disulfide bond may include replacement of an amino acid residue at a corresponding position of a phytase sequence to be introduced with the disulfide bond with an amino acid residue capable of forming a disulfide bond (for example, cysteine residue Cys, homocysteine residue Hey, etc.,); and/or insertion of an amino acid residue capable of forming a disulfide bond at the corresponding position. Such replacement and/or insertion may be, for example, achieved by a site-directed mutagenesis method well known in the art. “Introduction” also includes a situation where any one or two amino acid residues for forming the disulfide bonds are generated by natural mutation.

In some specific embodiments, the amino acid sequence of the variant is SEQ ID NO: 99, SEQ ID NO: 100, SEQ ID NO: 101, SEQ ID NO: 103, SEQ ID NO: 104, SEQ ID NO: 105, SEQ ID NO: 106, SEQ ID NO: 108, SEQ ID NO: 109, SEQ ID NO: 110, SEQ ID NO: 111, SEQ ID NO: 113, SEQ ID NO: 114, SEQ ID NO: 116, SEQ ID NO: 117 or SEQ ID NO: 118.

In addition, the present invention also provides a nucleic acid encoding the parent phytase variant as described above.

In addition, the present invention also provides a vector comprising the nucleic acid as described above.

In addition, the present invention also provides a host cell comprising the vector as described above.

In preferred embodiments, the host cell is a fungal cell, a bacterial cell or a plant cell.

Further preferably, the host cell is a fungal cell, and the fungal cell is selected from a Pichia pastoris cell or an Aspergillus niger cell.

In addition, the present invention also provides a method for producing the parent phytase variant as described above, comprising:
(a) culturing the host cell as described above under a condition suitable for expression of the parent phytase variant; and
(b) recovering the parent phytase variant.

The present invention has the following beneficial effects:

On the basis of phytase or a phytase mutant, one or more mutation sites as described in the present invention are introduced, especially multiple mutation sites are introduced at the same time, and the phytase variant obtained thereby has a residual activity increased by 16%-27% compared to the parent phytase after being treated in a water bath at 85° C. for 5 min. Therefore, the technical solution of the present invention can significantly improve the enzymatic activity of phytase, especially in terms of thermal resistance stability, steam resistance stability, and granulation stability, which is significantly better than the existing wild-type or mutant phytase.

### Nomenclature and Definition

Sequence identity: i.e., sequence identical, is defined as the percentage of amino acid residues in a candidate sequence that are identical to the amino acid residues in a specific peptide or polypeptide sequence, after aligning the sequences and introducing gaps, if necessary, to achieve the maximum percent sequence identity, and not considering any conservative substitutions as part of the sequence identity. Alignment of sequences for the purpose of determining percent amino acid sequence identity can be achieved by using a variety of methods within the skill in the art, for instance, using publicly available computer software, such as BLAST, BLAST-2, ALIGN or Megalign (DNASTAR) software. A person skilled in the art can determine appropriate parameters for measuring alignment, comprising any algorithms needed to achieve maximal alignment over the full length of the sequences being compared.

Parent: refers to a phytase that can produce the variant of the present invention after being subjected to the mutation described in the present invention. The parent may be a naturally occurring (wild-type) phytase or a mutant thereof prepared by a suitable method, or may also be an allelic variant.

Host cell: refers to any cell type susceptible to transformation, transfection, transduction, etc. with a vector comprising the nucleic acid of the present invention. The term “host cell” encompasses any progeny of a parent cell that is not identical to the parent cell due to mutations that occur during replication.

Substitution: refers to a replacement of an original amino acid occupying a position with a different amino acid.

Variant: refers to an enzyme that has phytase activity, and relative to the parent phytase thereof comprises one or more (several) amino acid residue substitutions at one or more (several) positions. Construction of the variant of the present invention can be achieved by culturing a host cell containing a nucleic acid sequence encoding the variant under a condition suitable for production of the variant, and then the variant can be recovered from the resulting culture solution.

Wild-type phytase: refers to a phytase expressed by a naturally occurring microorganism, such as a natural phytase derived from Escherichia coli found in nature.

When describing the phytase variant of the present invention, the following nomenclature is used for reference. In all cases, the recognized IUPAC one- or three-character amino acid abbreviations are used.

For amino acid substitutions, the following nomenclature is used: original amino acid, position, amino acid for substitution. For example, the substitution of threonine with alanine at position 226 is named “Thr226Ala” or “T226A”. Multiple mutations are separated by a plus sign (“+”), for example, “Gly205Arg+Ser411Phe” or “G205R+S411F” represents the substitution of glycine (G) with arginine (R) at position 205 and the substitution of serine(S) with phenylalanine (F) at position 411, respectively.

### BRIEF DESCRIPTION OF THE DRAWINGS

The specific embodiments of the present invention will be further described in detail below in conjunction with the drawings.

FIG. 1 shows a plasmid map of pPIC9K-APPA-Y0.

FIG. 2 shows detection results of thermal stability of the parent phytase and variants thereof in example 1.

FIG. 3 shows detection results of thermal stability of variants with site-directed saturation mutations on an amino acid at position 295 in example 3.

FIG. 4 shows detection results of thermal stability of variants with site-directed saturation mutations on an amino acid at position 349 in example 3.

FIG. 5 shows detection results of thermal stability of variants with site-directed saturation mutations on an amino acid at position 374 in example 3.

FIG. 6 shows detection results of thermal stability of variants with combinations of mutations in example 4.

FIG. 7 shows detection results of thermal stability of variants of parent phytase APPA-An1 in example 5.

FIG. 8 shows detection results of thermal stability of variants of parent phytase APPA-An5 in example 6.

FIG. 9 shows detection results of thermal stability of variants of parent phytase APPA-An10 in example 7.

FIG. 10 shows detection results of thermal stability of variants of parent phytase APPA-An15 in example 8.

FIG. 11 shows detection results of thermal stability of variants of parent phytase APPA-An18 in example 9.

### DETAILED DESCRIPTION OF EMBODIMENTS

In order to illustrate the present invention more clearly, the present invention is further described below in conjunction with preferred examples and the drawings. A person skilled in the art should understand that the content specifically described below is illustrative and not restrictive, and is not intended to limit the scope of protection of the present invention.

### Example 1 Construction of Mutant Expression Strains of Pichia pastoris

The present invention cites the amino acid sequence of the phytase mutant named APPA-M2-O in patent WO 2019228441, and the amino acid sequence thereof is as shown in SEQ ID NO: 2 of the present invention. This phytase mutant is named as APPA-Y0 in the present invention. APPA-Y0 is a phytase mutant obtained by subjecting Escherichia coli-derived wild-type phytase (the amino acid sequence thereof is as shown in SEQ ID NO: 1) to mutation, screening, glycosylation and introduction of disulfide bond sites, and the specific method is as described in WO 2019228441. The mutant has excellent thermal stability. This example involves further mutation and screening on the basis of the APPA-Y0 sequence.

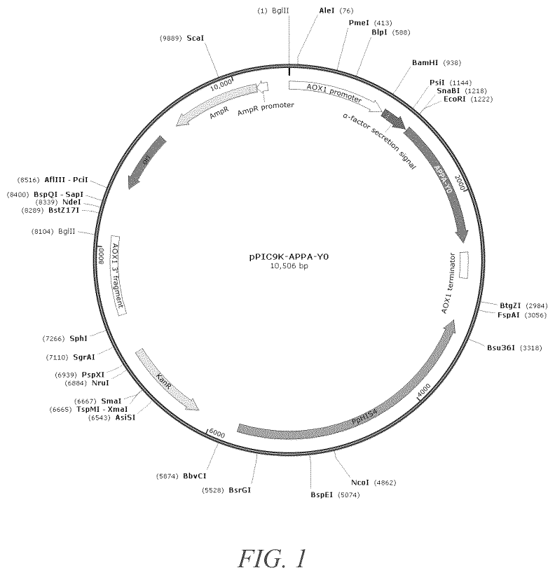

The nucleic acid sequence of APPA-Y0 expressed in Pichia pastoris was synthesized by GenScript Biotech Co., Ltd., and the gene was cloned into a Pichia pastoris vector, wherein the expression vector was pPIC9K, and the Alpha factor of Saccharomyces cerevisiae was used as a signal peptide. The phytase expression plasmid pPIC9K-APPA-Y0 is as shown in FIG. 1.

In the first round of variant screening, APPA-Y0 was used as a parent phytase. In order to improve the thermal stability of the parent phytase, the inventors designed the following 37 mutants on the basis of the amino acid sequence and protein structure analysis of APPA-Y0, as shown in Table 1. In the design, each variant comprised one amino acid substitution relative to the parent phytase APPA-Y0, and these variants were named APPA-Y1 to APPA-Y37 with amino acid sequences as shown in SEQ ID NOs: 3-39, respectively.

TABLE 1
Phytase variants constructed in the first round

| | Mutation site | Sequence number |
|---|---|---|
| Mutant name | | |
| APPA-Y0 | Parent | SEQ ID NO: 2 |
| APPA-Y1 | A25P | SEQ ID NO: 3 |
| APPA-Y2 | M29L | SEQ ID NO: 4 |
| APPA-Y3 | K43P | SEQ ID NO: 5 |
| APPA-Y4 | R50K | SEQ ID NO: 6 |
| APPA-Y5 | G52A | SEQ ID NO: 7 |
| APPA-Y6 | L58M | SEQ ID NO: 8 |
| APPA-Y7 | D69Q | SEQ ID NO: 9 |
| APPA-Y8 | G97A | SEQ ID NO: 10 |
| APPA-Y9 | E98Q | SEQ ID NO: 11 |
| APPA-Y10 | A105F | SEQ ID NO: 12 |
| APPA-Y11 | A109D | SEQ ID NO: 13 |
| APPA-Y12 | T118Q | SEQ ID NO: 14 |
| APPA-YI3 | S120T | SEQ ID NO: 15 |
| APPA-Y14 | P121M | SEQ ID NO: 16 |
| APPA-Y15 | N137I | SEQ ID NO: 17 |
| APPA-Y16 | T156M | SEQ ID NO: 18 |
| APPA-Y17 | R159L | SEQ ID NO: 19 |
| APPA-Y18 | T191P | SEQ ID NO: 20 |
| Sequence name | | |
| APPA-Y19 | V211L | SEQ ID NO: 21 |
| APPA-Y20 | Q225Y | SEQ ID NO: 22 |
| APPA-Y21 | S240W | SEQ ID NO: 23 |
| APPA-Y22 | N244Y | SEQ ID NO: 24 |
| APPA-Y23 | Q258F | SEQ ID NO: 25 |
| APPA-Y24 | S266Y | SEQ ID NO: 26 |
| APPA-Y25 | T295P | SEQ ID NO: 27 |
| APPA-Y26 | S296K | SEQ ID NO: 28 |
| APPA-Y27 | I300L | SEQ ID NO: 29 |
| APPA-Y28 | L307I | SEQ ID NO: 30 |
| APPA-Y29 | Q346F | SEQ ID NO: 31 |
| APPA-Y30 | Q349K | SEQ ID NO: 32 |
| APPA-Y31 | L352Y | SEQ ID NO: 33 |
| APPA-Y32 | F354Y | SEQ ID NO: 34 |
| APPA-Y33 | M360L | SEQ ID NO: 35 |
| APPA-Y34 | K363A | SEQ ID NO: 36 |
| APPA-Y35 | T370C | SEQ ID NO: 37 |
| APPA-Y36 | E374R | SEQ ID NO: 38 |
| APPA-Y37 | T397I | SEQ ID NO: 39 |

The variant plasmids were named pPIC9K-APPA-Y1 to pPIC9K-APPA-Y37 according to the variant names in the table above. In order to express the parent phytase and the variants thereof, Pichia pastoris GS115 and plasmids were manipulated by using the Pichia expression kit (Invitrogen) with reference to specifications thereof. Specifically, Pichia pastoris GS115 strains were subjected to a plate culture at 30° C. for 48 h by using a YPD medium (1% yeast extract, 2% protein, 2% glucose, and 1.5% agar), and then single clones were picked to 4 mL of a YPD liquid medium (1% yeast extract, 2% protein, and 2% glucose), cultured at 30° C. at 200 rpm for 12 h, transferred to an Erlenmeyer flask containing 30 mL of a YPD liquid medium, and cultured at 30° C. at 220 rpm for 4-5 h. After an OD600 value was detected to be in a range of 1.1-1.3, a culture solution was centrifuged at 4° C. at 9,000 rpm for 2 min. 4 mL of thalli were respectively collected into sterile EP tubes. Supernatants were gently removed. Residual supernatants were thoroughly absorbed by a sterile filter paper. Then the thalli were resuspended in 1 mL of pre-cooled sterile water and centrifuged at 4° C. at 9,000 rpm for 2 min, and supernatants were removed. The above steps were repeated. The thalli were resuspended in 1 mL of pre-cooled sorbitol (1 mol/L). Centrifugation at 4° C. at 9,000 rpm was performed for 2 min. Supernatants were removed. The thalli were resuspended in 100-150 μl of pre-cooled sorbitol (1 mol/L). Hereto, preparation of competent cells was completed. The expression plasmid pPIC9K-APPA-Y0 and other 37 variants were linearized with BglII. Linearized fragments were purified and recovered, and then transformed into the above-mentioned Pichia pastoris GS115 competent cells by an electroporation method. The mixture was evenly plated on an MD plate, which was inverted and cultured at 30° C. for 2-3 days. All the colonies were washed off the plate by sterile water, and then plated on a YPD (0.5 mg/mL-8 mg/mL) plate containing different concentrations of geneticin for screening multiple copies of transformants. The recombinant strains of Pichia pastoris were obtained by screening on the MD plate and were named as APPA-Y0 and APPA-Y1 to APPA-Y37. The above-mentioned clones obtained by screening were respectively transferred to BMGY mediums, cultured in a shaker at 30° C. at 250 rpm for 24 h, then transferred to BMMY mediums, and maintained at 30° C., 250 rpm, and 0.5% methanol was added every day to induce expression for 120 h. The thalli were removed by centrifuging at 9000-12000 rpm for 10 min to obtain fermentation supernatants containing phytase APPA-Y0 and other 37 variants. The results of SDS-PAGE shown that four variants, APPA-Y1, APPA-Y7, APPA-Y8 and APPA-Y16, were not expressed, and the remaining APPA-Y0 and other 33 variants were expressed.

### Example 2 Determination of Thermal Stability

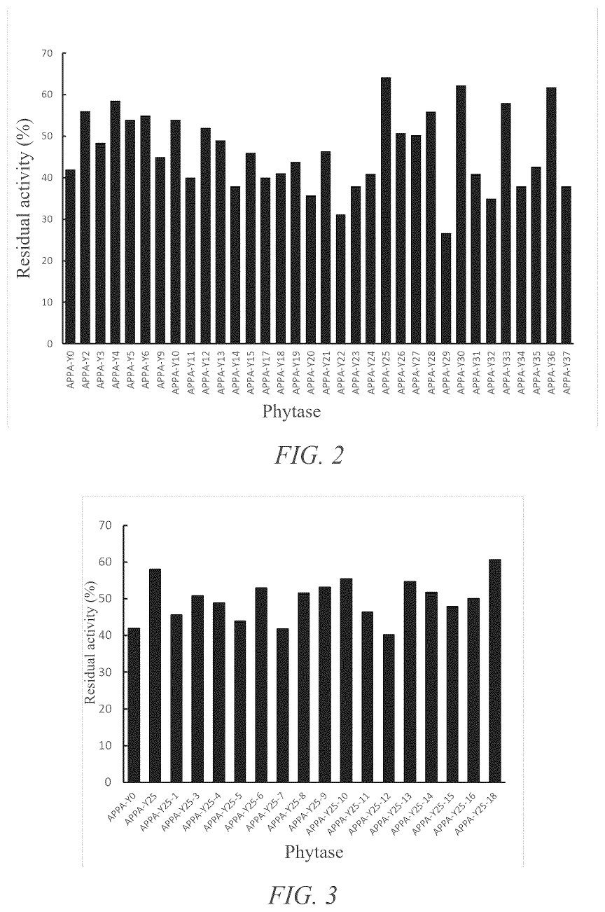

Phytase activity determination conforms to the standards of GBT18634-2009 document. The 33 variant samples and the parent phytase APPA-Y0 in example 1 were diluted to 100 U/mL with water. 9 mL of water was added to a 25 mL colorimetric tube; 1 mL of an enzyme sample was pipetted with a pipette, quickly added to the test tube and mixed quickly with a mixer, and the test tube was placed in a constant temperature water bath at 85° C. for exactly 5 min. The samples were quickly cooled to room temperature and diluted with water. The residual activity of each sample was determined, so as to calculate the residual activity of the enzyme at different treatment temperatures. The enzyme activity before thermal treatment was set as 100%, and the obtained thermal stability data are as shown in FIG. 2.

According to the above-mentioned experimental results, the introduction of mutation sites hada significant effect on phytase.

The introduction of some mutation sites resulted in abnormal expression of phytase, such as APPA-Y1, APPA-Y7, APPA-Y8 and APPA-Y16;
the introduction of some mutation sites may result in reduced thermal stability of phytase, for example, the thermal stability of APPA-Y22 and APPA-Y29 was significantly lower than that of APPA-Y0;
and the introduction of some mutation sites also resulted in significantly improved thermal resistance of phytase, such as APPA-Y2 (M29L), APPA-Y4 (R50K), APPA-Y5 (G52A), APPA-Y6 (L58M), APPA-Y10 (A105F), APPA-Y12 (T118Q), APPA-Y25 (T295P), APPA-Y28 (L3071), APPA-Y30 (Q349K), APPA-Y33 (M360L), and APPA-Y36 (E374R), wherein APPA-Y25 (T295P), APPA-Y30 (Q349K), and APPA-Y36 (E374R) had the best performance, with about 19%-22% improved residual activity of the variants compared to the parent phytase APPA-Y0.

### Example 3 Saturation Mutation on 3 Mutation Sites in Example 2

The 3 mutation sites T295P, Q349K and E374R corresponding to 3 mutant strains APPA-Y25, APPA-Y30, and APPA-Y36 with the most improved thermal resistance in example 2 were subjected to saturation mutation, that is, the amino acids at positions 295, 349 and 374 were mutated into other 18 amino acids. The corresponding sequence names were as shown in tables 2-4 below.

TABLE 2
Site-directed saturation mutation on amino acid at position 295

| Mutant name | Mutation site | Sequence number |
|---|---|---|
| APPA-Y25-1 | T295A | SEQ ID NO: 44 |
| APPA-Y25-2 | T295C | SEQ ID NO: 45 |
| APPA-Y25-3 | T295D | SEQ ID NO: 46 |
| APPA-Y25-4 | T295E | SEQ ID NO: 47 |
| APPA-Y25-5 | T295F | SEQ ID NO: 48 |
| APPA-Y25-6 | T295G | SEQ ID NO: 49 |
| APPA-Y25-7 | T295H | SEQ ID NO: 50 |
| APPA-Y25-8 | T295I | SEQ ID NO: 51 |
| APPA-Y25-9 | T295K | SEQ ID NO: 52 |
| APPA-Y25-10 | T295L | SEQ ID NO: 53 |
| APPA-Y25-11 | T295M | SEQ ID NO: 54 |
| APPA-Y25-12 | T295N | SEQ ID NO: 55 |
| APPA-Y25-13 | T295Q | SEQ ID NO: 56 |
| APPA-Y25-14 | T295R | SEQ ID NO: 57 |
| APPA-Y25-15 | T295S | SEQ ID NO: 58 |
| APPA-Y25-16 | T295V | SEQ ID NO: 59 |
| APPA-Y25-17 | T295W | SEQ ID NO: 60 |
| APPA-Y25-18 | T295Y | SEQ ID NO: 61 |

TABLE 3
Site-directed saturation mutation on amino acid at position 349

| Mutant name | Mutation site | Sequence number |
|---|---|---|
| APPA-Y30-1 | Q349A | SEQ ID NO: 62 |
| APPA-Y30-2 | Q349C | SEQ ID NO: 63 |
| APPA-Y30-3 | Q349D | SEQ ID NO: 64 |
| APPA-Y30-4 | Q349E | SEQ ID NO: 65 |
| APPA-Y30-5 | Q349F | SEQ ID NO: 66 |
| APPA-Y30-6 | Q349G | SEQ ID NO: 67 |
| APPA-Y30-7 | Q349H | SEQ ID NO: 68 |
| APPA-Y30-8 | Q349I | SEQ ID NO: 69 |
| APPA-Y30-9 | Q349L | SEQ ID NO: 70 |
| APPA-Y30-10 | Q349M | SEQ ID NO: 71 |
| APPA-Y30-11 | Q349N | SEQ ID NO: 72 |
| APPA-Y30-12 | Q349P | SEQ ID NO: 73 |
| APPA-Y30-13 | Q349R | SEQ ID NO: 74 |
| APPA-Y30-14 | Q349S | SEQ ID NO: 75 |
| APPA-Y30-15 | Q349T | SEQ ID NO: 76 |
| APPA-Y30-16 | Q349V | SEQ ID NO: 77 |
| APPA-Y30-17 | Q349W | SEQ ID NO: 78 |
| APPA-Y30-18 | Q349Y | SEQ ID NO: 79 |

TABLE 4
Site-directed saturation mutation on amino acid at position 374

| Mutant name | Mutation site | Sequence number |
|---|---|---|
| APPA-Y36-1 | E374A | SEQ ID NO: 80 |
| APPA-Y36-2 | E374C | SEQ ID NO: 81 |
| APPA-Y36-3 | E374D | SEQ ID NO: 82 |
| APPA-Y36-4 | E374F | SEQ ID NO: 83 |
| APPA-Y36-5 | E374G | SEQ ID NO: 84 |
| APPA-Y36-6 | E374H | SEQ ID NO: 85 |
| APPA-Y36-7 | E374I | SEQ ID NO: 86 |
| APPA-Y36-8 | E374K | SEQ ID NO: 87 |
| APPA-Y36-9 | E374L | SEQ ID NO: 88 |
| APPA-Y36-10 | E374M | SEQ ID NO: 89 |
| APPA-Y36-11 | E374N | SEQ ID NO: 90 |
| APPA-Y36-12 | E374P | SEQ ID NO: 91 |
| APPA-Y36-13 | E374Q | SEQ ID NO: 92 |
| APPA-Y36-14 | E374S | SEQ ID NO: 93 |
| APPA-Y36-15 | E374T | SEQ ID NO: 94 |
| APPA-Y36-16 | E374V | SEQ ID NO: 95 |
| APPA-Y36-17 | E374W | SEQ ID NO: 96 |
| APPA-Y36-18 | E374Y | SEQ ID NO: 97 |

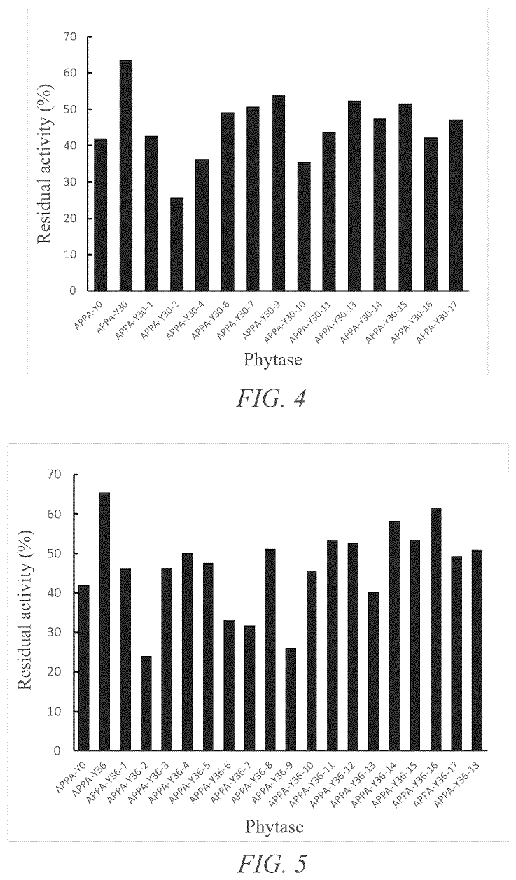

According to the method in example 1, each variant was expressed using Pichia pastoris, and then the thermal stability of each variant was determined according to the method in example 2. The results were as shown in FIG. 3-FIG. 5.

The results in the figures showed that performing saturation mutation on amino acids at 3 positions respectively had a significant effect on the variant.

Some mutations even resulted in abnormal expression, such as APPA-Y25-2, APPA-Y25-17, APPA-Y30-3, APPA-Y30-5, APPA-Y30-8, APPA-Y30-12 and APPA-Y30-18;
in addition, the introduction of some mutation sites may result in reduced thermal stability of phytase, for example, the thermal stability of APPA-Y30-2, APPA-Y36-2 and APPA-Y36-9 was significantly lower than that of APPA-Y0;
and furthermore, the introduction of some mutation sites significantly improved thermal resistance of phytase, such as APPA-Y25-6 (T295G), APPA-Y25-9 (T295K), APPA-Y25-10 (T295L), APPA-Y25-13 (T295Q), APPA-Y25-18 (T295Y), APPA-Y30-9 (Q349L), APPA-Y30-6 (Q349G), APPA-Y30-7 (Q349H), APPA-Y30-13 (Q349R), APPA-Y30-15 (Q349T), APPA-Y36-11 (E374N), APPA-Y36-14 (E374S), APPA-Y36-15 (E374T), APPA-Y36-16 (E374V), APPA-Y36-4 (E374F), APPA-Y36-8 (E374K), APPA-Y36-12 (E374P), and APPA-Y36-18 (E374Y).

It can be seen from the experimental data of example 2 and example 3 that, when a single amino acid substitution was introduced on the basis of the parent phytase, the introduction of mutation sites T295P, Q349K, E374R, T295Y, Q349L or E374V provided better effects, which significantly improved the thermal stability of phytase.

### Example 4 Combinations of Mutation Sites

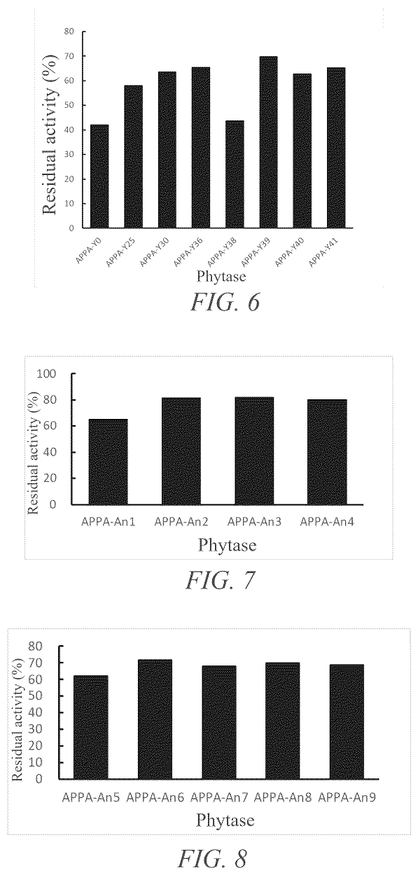

The 3 mutant sites T295P, Q349K and E374R corresponding to the 3 mutant strains APPA-Y25, APPA-Y30 and APPA-Y36 in example 2 were combined, and the corresponding variants were constructed according to the method in example 1 and expressed in Pichia pastoris. The specific mutations were as shown in table 5 below. The thermal stability was determined according to the detection method in example 2. The obtained thermal stability data were as shown in FIG. 6. It can be seen from the detection results that the phytase variants subjected to the combined mutations had significantly improved thermal resistance compared with the parent phytase APPA-Y0. The combination of some mutations increased thermal resistance of the variants by 16%-27% compared with APPA-Y0, indicating that a suitable combination can create a more stable mutant, which can be expected to have good performance in feed granulation.

TABLE 5
Phytase variants subjected to combinations of mutations

| Sequence name | Mutation site | Sequence number |
|---|---|---|
| APPA-Y38 | T295P + Q349K | SEQ ID NO: 40 |
| APPA-Y39 | T295P + E374R | SEQ ID NO: 41 |
| APPA-Y40 | Q349K + E374R | SEQ ID NO: 42 |
| APPA-Y41 | T295P + Q349K + E374R | SEQ ID NO: 43 |

### Example 5 Expression of Variants of Parent Phytase APPA-An1 in Aspergillus niger

APPA-An1 is another mutant having excellent thermal resistance obtained by mutation of wild-type phytase from Escherichia coli, and the amino acid sequence thereof was as shown in SEQ ID NO: 98.

QSEEELKLESVVIVSRHGVRAPTKFTQLMQDVTPYAWPTWPVKLGELTP
RGGELIAYLGHYWRQRLVADELLPNQTCPQPGQVAIIADVDERTRKTGE
AFAAGLAPGCAITVHHQADTSSPDPLFNPLKTGVCQLDVARVTRAILER
AGGSIADFTNHYQPAFRELERVLNFSQSPLCKNREKQNEPCSLTQALPS
ELKVSADNVSLTGAWSLASMLTEIFLLQQAQGMPEPGWGRITDSHQWNT
LLSLHNAYFDLLQRTPEVARSAATPLLDLIKTALTPNGTQKSAYGVTLP
TSVLFIAGHDTNLANLGGALELNWTLPGQPDNYPPGGELVFERWRRLSD
NSCWIQVSLVFQTLQQMRDKTPLSLNTPPGEVKLTLPGCEERNAQGMCS
CAGFTQIVNEARIPACSL*

In order to test whether the mutation sites discovered in the present invention can also function on the existing phytase mutants and further improve the stability of phytase, this example involved using APPA-An1 as a parent phytase, adding amino acid substitutions Q349K and E374R, alone or in combination, to the amino acid sequence of APPA-An1, wherein each mutant was named according to APPA-An2 to APPA-An4, as shown in the table below. The above-mentioned phytase mutants were expressed in Aspergillus niger according to the method described in patent application CN 107353327 A. After the supernatant from the shake flask was obtained, the thermal stability was determined as described in example 2, and incubation was performed at 85° C. for 5 minutes. The experimental results were as shown in FIG. 7. The experimental results showed that the 3 variants all showed a significant improvement in stability performance and demonstrate more excellent stability than the parent phytase APPA-An1, that is, a phytase variant with a higher stability can be obtained by introducing 1-2 mutation sites of the present invention into the parent phytase, and can be expected to have good performance in feed granulation.

TABLE 6
Variants of parent APPA-An1

| Mutant name | Mutation site | Sequence number |
|---|---|---|
| APPA-An1 | Parent | SEQ ID NO: 98 |
| APPA-An2 | Q349K | SEQ ID NO: 99 |
| APPA-An3 | E374R | SEQ ID NO: 100 |
| APPA-An4 | Q349K + E374R | SEQ ID NO: 101 |

### Example 6 Expression of Variants of Parent Phytase APPA-An5 in Aspergillus niger

APPA-An5 is another mutant having excellent thermal resistance obtained by mutation of wild-type phytase, and the amino acid sequence thereof was as shown in SEQ ID NO: 102. In order to test whether the mutation sites discovered in the present invention can also function on the existing phytase mutants and further improve the stability of phytase, this example involved using APPA-An5 as a parent phytase, adding T295P, Q349K and E374R, alone or in combination, to the amino acid sequence of APPA-An5, wherein each mutant was named according to APPA-An6 to APPA-An9, as shown in table 7. The above-mentioned phytase variants were expressed in Aspergillus niger according to the method in example 5. After the supernatant from the shake flask was obtained, the thermal stability was determined as described in example 2, and incubation was performed at 85° C. for 5 minutes. The experimental results were as shown in FIG. 8. It has been found that the 4 mutants all showed a significant improvement in stability performance and demonstrated more excellent stability than the parent APPA-An5, that is, a phytase mutant with a higher stability can be obtained by introducing 1-2 mutation sites of the present invention into the parent phytase, and can be expected to have good performance in feed granulation.

TABLE 7
Phytase mutants

| Mutant name | Mutation site | Sequence number |
|---|---|---|
| APPA-An5 | Parent | SEQ ID NO: 102 |
| APPA-An6 | T295P | SEQ ID NO: 103 |
| APPA-An7 | Q349K | SEQ ID NO: 104 |
| APPA-An8 | E374R | SEQ ID NO: 105 |
| APPA-An9 | Q349K + E374R | SEQ ID NO: 106 |

### Example 7 Expression of Variants of Parent Phytase APPA-An10 in Aspergillus niger

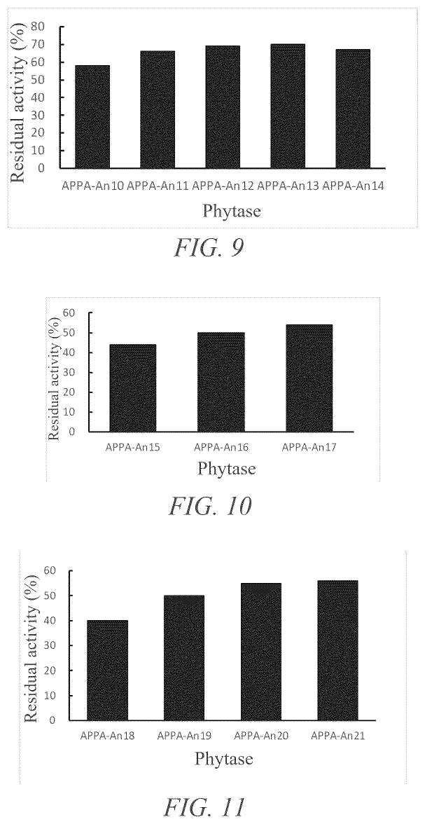

APPA-An10 is another mutant having excellent thermal resistance obtained by mutation of wild-type phytase and screening, and the amino acid sequence thereof was as shown in SEQ ID NO: 107. In order to test whether the mutation sites discovered in the present invention can also function on the existing phytase mutants and further improve the stability of phytase, this example involved using APPA-An10 as a parent phytase, adding T295P, Q349K and E374R, alone or in combination, to the amino acid sequence of APPA-An10, wherein each mutant was named according to APPA-An11 to APPA-An14, as shown in table 8. The above-mentioned phytase variants were expressed in Aspergillus niger according to the method in example 5. After the supernatant from the shake flask was obtained, the thermal stability was determined as described in example 2, and incubation was performed at 85° C. for 5 minutes. The experimental results were as shown in FIG. 9. It has been found that the 4 mutants all showed a significant improvement in stability performance and demonstrate more excellent stability than the parent APPA-An10, that is, a phytase mutant with a higher stability can be obtained by introducing 1-3 mutation sites of the present invention into the parent phytase, and can be expected to have good performance in feed granulation.

TABLE 8
Phytase mutants

| Mutant name | Mutation site | Sequence number |
|---|---|---|
| APPA-An10 | Parent | SEQ ID NO: 107 |
| APPA-An11 | E374R | SEQ ID NO: 108 |
| APPA-An12 | T295P + Q349K | SEQ ID NO: 109 |
| APPA-An13 | T295P + E374R | SEQ ID NO: 110 |
| APPA-An14 | T295P + Q349K + E374R | SEQ ID NO: 111 |

### Example 8 Expression of Variants of Parent Phytase APPA-An15 in Aspergillus niger

APPA-An15 is another mutant having excellent thermal resistance obtained by mutation of wild-type phytase and screening, and the amino acid sequence thereof was as shown in SEQ ID NO: 112. In order to test whether the mutation sites discovered in the present invention can also function on the existing phytase mutants and further improve the stability of phytase, this example involved using APPA-An15 as a parent phytase, adding T295P and E374R separately to the amino acid sequence of APPA-An15, wherein each mutant was named according to APPA-An16 to APPA-An17, as shown in the table below. The above-mentioned phytase variants were expressed in Aspergillus niger according to the method in example 5. After the supernatant from the shake flask was obtained, the thermal stability was determined as described in example 2, and incubation was performed at 85° C. for 5 minutes. The experimental results were as shown in FIG. 10. It has been found that the 2 mutants all showed a significant improvement in stability performance and demonstrated more excellent stability than the parent APPA-An15, that is, a phytase mutant with a higher stability can be obtained by introducing 1 mutation site of the present invention into the parent phytase, and can be expected to have good performance in feed granulation.

TABLE 9
Phytase mutants

| Mutant name | Mutation site | Sequence number |
|---|---|---|
| APPA-An15 | Parent | SEQ ID NO: 112 |
| APPA-An16 | T295P | SEQ ID NO: 113 |
| APPA-An17 | E374R | SEQ ID NO: 114 |

### Example 9 Expression of Variants of Parent Phytase APPA-An18 in Aspergillus niger

APPA-An18 is another mutant having excellent thermal resistance obtained by mutation of wild-type phytase and screening, and the amino acid sequence thereof was as shown in SEQ ID NO: 115. In order to test whether the mutation sites discovered in the present invention can also function on the existing phytase mutants and further improve the stability of phytase, this example involved using APPA-An18 as a parent phytase, adding T295P, Q349K and E374R, alone or in combination, to the amino acid sequence of APPA-An18, wherein each mutant was named according to APPA-An19 to APPA-An21, as shown in table 10. The above-mentioned phytase variants were expressed in Aspergillus niger according to the method in example 5. After the supernatant from the shake flask was obtained, the thermal stability was determined as described in example 2, and incubation was performed at 85° C. for 5 minutes. The experimental results were as shown in FIG. 11. It has been found that the 3 mutants all showed a significant improvement in stability performance and demonstrated more excellent stability than the parent APPA-An18, that is, a phytase mutant with a higher stability can be obtained by introducing 1-3 mutation sites of the present invention into the parent phytase, and can be expected to have good performance in feed granulation.

TABLE 10
Phytase mutants

| Mutant name | Mutation site | Sequence number |
|---|---|---|
| APPA-An18 | Parent | SEQ ID NO: 115 |
| APPA-An19 | Q349K | SEQ ID NO: 116 |
| APPA-An20 | Q349K + E374R | SEQ ID NO: 117 |
| APPA-An21 | T295P + Q349K + E374R | SEQ ID NO: 118 |

Obviously, the above-mentioned examples of the present invention are merely examples used for clearly describing the present invention, instead of limiting the implementations of the present invention. For a person of ordinary skill in the art, it would also be possible to make other different forms of changes or variations on the basis of the above-mentioned description, and it is not possible to exhaust all embodiments here. Any obvious changes or variations derived from the technical solution of the present invention are still within the scope of protection of the present invention.

What is claimed is:

1. A thermostable phytase variant, which
(a) has one or more amino acid substitutions at positions corresponding to positions 295, 349, and 374 of the polypeptide of SEQ ID NO: 1, wherein the amino acid position corresponding to position 295 of the polypeptide of SEQ ID NO: 1 is substituted with P, Y, G, K, L or Q, or wherein the amino acid position corresponding to position 349 of the polypeptide of SEQ ID NO:1 is substituted with K, L, G, H, R, or T;
(b) has at least 90% sequence identity to the polypeptide of SEQ ID NO: 98; and
(c) has phytase activity.

2. The phytase variant of claim 1, wherein the phytase variant is obtained by introducing one or more substitutions at positions corresponding to positions 295, 349, and 374 of the polypeptide of SEQ ID NO: 1 to a parent phytase, wherein the parent phytase has at least 90% sequence identity to the polypeptide of SEQ ID NO: 98.

3. The phytase variant of claim 2, wherein the amino acid sequence of the parent phytase is selected from: SEQ ID NO: 2, SEQ ID NO: 98, SEQ ID NO: 102, SEQ ID NO: 107, SEQ ID NO: 112, or SEQ ID NO: 115.

4. The phytase variant of claim 3, wherein the amino acid at the position corresponding to position 295 of the polypeptide of SEQ ID NO: 1 is substituted with P, Y, G, K, L or Q.

5. The phytase variant of claim 3, wherein the amino acid at the position corresponding to position 374 of the polypeptide of SEQ ID NO: 1 is substituted with R, V, N, S, T, F, K, P or Y.

6. The phytase variant of claim 3, wherein the amino acid at the position corresponding to position 295 of the polypeptide of SEQ ID NO: 1 is substituted with P, Y, G, K, L or Q, and wherein the amino acid at the position corresponding to position 374 of the polypeptide of SEQ ID NO: 1 is substituted with R, V, N, S, T, F, K, P or Y.

7. The phytase variant of claim 6, wherein the amino acid at the position corresponding to position 349 of the polypeptide of SEQ ID NO: 1 is substituted with K, L, G, H, R or T.

8. The phytase variant of claim 1, wherein the variant comprises an amino acid substitution or combination of substitutions that correspond to substitutions in the polypeptide of SEQ ID NO:1 selected from the group consisting of:
$$T 295 P ; Q 349 K ; E 374 R ; T 2 \; 95 Y ; Q 349 L ; E 3 \; 74 V ;$$
$$T 295 P + Q 349 K ; T 295 P + E 3 \; 74 R ; T 295 P + Q 349 L ; T 295 P + E 3 \; 74 V ;$$
$$T 295 Y + Q 349 K ; T 295 Y + E 374 R ; T 295 Y + Q 349 L ; T 295 Y + E 3 \; 74 V ;$$
$$Q 349 L + E 374 R ; Q 349 K + E 374 R ; Q 349 L + E 3 \; 74 V ; Q 349 K + E 3 \; 74 V ;$$
$$T 295 P + Q 349 K + E 374 R ; T 295 Y + Q 349 K + E 3 \; 74 R ;$$
$$T 295 P + Q 349 L + E 3 \; 74 R ; T 295 Y + Q 349 L + E 3 \; 74 R ;$$
$$T 295 P + Q 349 K + E 3 \; 74 V ; T 295 Y + Q 349 K + E 3 \; 74 V ;$$
$$T 295 P + Q 349 L + E 3 \; 74 V ; and T 295 Y + Q 349 L + E 374 V .$$

9. The phytase variant of claim 1, wherein the variant further comprises at least one pair of introduced disulfide bonds.

10. The phytase variant of claim 9, wherein the variant comprises disulfide bonds formed between an amino acid residue at a position corresponding to position 346 of the polypeptide of SEQ ID NO: 1 and an amino acid residue at a position corresponding to position 393 of the polypeptide of SEQ ID NO: 1.

11. The phytase variant of claim 1, wherein the variant comprises a polypeptide having at least 90%, 91%, 92%, 93%, 94%, 95%, 96%, 97%, 98%, 99%, or 100% identity to an amino acid sequence selected from SEQ ID NO: 99, SEQ ID NO: 100, SEQ ID NO: 101, SEQ ID NO: 103, SEQ ID NO: 104, SEQ ID NO: 105, SEQ ID NO: 106, SEQ ID NO: 108, SEQ ID NO: 109, SEQ ID NO: 110, SEQ ID NO: 111, SEQ ID NO: 113, SEQ ID NO: 114, SEQ ID NO: 116, SEQ ID NO: 117, and SEQ ID NO: 118.

12. The phytase variant of claim 1, wherein the variant comprises a polypeptide of SEQ ID NO: 105, SEQ ID NO: 106, SEQ ID NO: 108, SEQ ID NO: 109, or SEQ ID NO: 110.


SEQUENCE LISTING (118 sequences)

SEQ ID NO: 1 - protein, 410 aa, Escherichia coli
QSEPELKLESVVIVSRHGVRAPTKATQLMQDVTPDAWPTWPVKLGWLTPRGGELIAYLGHYQRQRLVADGLLAKKGCPQSGQVAIIADVDERTRKTGEAFAAGLAPDCAITVHTQADTSSPDPLFNPLKTGVCQLDNANVTDAILSRAGGSIADFTGHRQTAFRELERVLNFPQSNLCLKREKQDESCSLTQALPSELKVSADNVSLTGAVSLASMLTKIFLLQQAQGMPEPGWGRITDSHQWNTLLSLHNAQFYLLQRTPEVARSRATPLLDLIKTALTPHPPQKQAYGVTLPTSVLFIAGHDTNLANLGGALELNWTLPGQPDNTPPGGELVFERWRRLSDNSQWIQVSLVFQTLQQMRDKTPLSLNTPPGEVKLTLAGCEERNAQGMCSLAGFTQIVNEARIPACSL

SEQ ID NO: 2 - protein, 410 aa, synthetic construct
  REGION 1..410: note = APPA-Y0
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIQVSLVFQTLQQMRDKTPLSLNTPPGEVKLTLAGCEERNAQGMCSCAGFTQIVNEARIPACSL

SEQ ID NO: 3 - protein, 410 aa, synthetic construct
  REGION 1..410: note = APPA-Y1
QSEPELKLESVVIVSRHGVRAPTKPTQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIQVSLVFQTLQQMRDKTPLSLNTPPGEVKLTLAGCEERNAQGMCSCAGFTQIVNEARIPACSL

SEQ ID NO: 4 - protein, 410 aa, synthetic construct
  REGION 1..410: note = APPA-Y2
QSEPELKLESVVIVSRHGVRAPTKATQLLQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIQVSLVFQTLQQMRDKTPLSLNTPPGEVKLTLAGCEERNAQGMCSCAGFTQIVNEARIPACSL

SEQ ID NO: 5 - protein, 410 aa, synthetic construct
  REGION 1..410: note = APPA-Y3
QSEPELKLESVVIVSRHGVRAPTKATQLMQDVTPDAWPTWPVPLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIQVSLVFQTLQQMRDKTPLSLNTPPGEVKLTLAGCEERNAQGMCSCAGFTQIVNEARIPACSL

SEQ ID NO: 6 - protein, 410 aa, synthetic construct
  REGION 1..410: note = APPA-Y4
QSEPELKLESVVIVSRHGVRAPTKATQLMQDVTPDAWPTWPVKLGELTPKGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIQVSLVFQTLQQMRDKTPLSLNTPPGEVKLTLAGCEERNAQGMCSCAGFTQIVNEARIPACSL

SEQ ID NO: 7 - protein, 410 aa, synthetic construct
  REGION 1..410: note = APPA-Y5
QSEPELKLESVVIVSRHGVRAPTKATQLMQDVTPDAWPTWPVKLGELTPRGAELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIQVSLVFQTLQQMRDKTPLSLNTPPGEVKLTLAGCEERNAQGMCSCAGFTQIVNEARIPACSL

SEQ ID NO: 8 - protein, 410 aa, synthetic construct
  REGION 1..410: note = APPA-Y6
QSEPELKLESVVIVSRHGVRAPTKATQLMQDVTPDAWPTWPVKLGELTPRGGELIAYMGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIQVSLVFQTLQQMRDKTPLSLNTPPGEVKLTLAGCEERNAQGMCSCAGFTQIVNEARIPACSL

SEQ ID NO: 9 - protein, 410 aa, synthetic construct
  REGION 1..410: note = APPA-Y7
QSEPELKLESVVIVSRHGVRAPTKATQLMQDVTPDAWPTWPVKLGELTPRGGELIAYLGHYWRQRLVAQELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIQVSLVFQTLQQMRDKTPLSLNTPPGEVKLTLAGCEERNAQGMCSCAGFTQIVNEARIPACSL

SEQ ID NO: 10 - protein, 410 aa, synthetic construct
  REGION 1..410: note = APPA-Y8
QSEPELKLESVVIVSRHGVRAPTKATQLMQDVTPDAWPTWPVKLGELTPRGGELIAYLGHYWRQRLVADELLPNQTCPQSGQVAIIADVDERTRKTAEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIQVSLVFQTLQQMRDKTPLSLNTPPGEVKLTLAGCEERNAQGMCSCAGFTQIVNEARIPACSL

SEQ ID NO: 11 - protein, 410 aa, synthetic construct
  REGION 1..410: note = APPA-Y9
QSEPELKLESVVIVSRHGVRAPTKATQLMQDVTPDAWPTWPVKLGELTPRGGELIAYLGHYWRQRLVADELLPNQTCPQSGQVAIIADVDERTRKTGQAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIQVSLVFQTLQQMRDKTPLSLNTPPGEVKLTLAGCEERNAQGMCSCAGFTQIVNEARIPACSL

SEQ ID NO: 12 - protein, 410 aa, synthetic construct
  REGION 1..410: note = APPA-Y10
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLFPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIQVSLVFQTLQQMRDKTPLSLNTPPGEVKLTLAGCEERNAQGMCSCAGFTQIVNEARIPACSL

SEQ ID NO: 13 - protein, 410 aa, synthetic construct
  REGION 1..410: note = APPA-Y11
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCDITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIQVSLVFQTLQQMRDKTPLSLNTPPGEVKLTLAGCEERNAQGMCSCAGFTQIVNEARIPACSL

SEQ ID NO: 14 - protein, 410 aa, synthetic construct
  REGION 1..410: note = APPA-Y12
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADQSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIQVSLVFQTLQQMRDKTPLSLNTPPGEVKLTLAGCEERNAQGMCSCAGFTQIVNEARIPACSL

SEQ ID NO: 15 - protein, 410 aa, synthetic construct
  REGION 1..410: note = APPA-Y13
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSTPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIQVSLVFQTLQQMRDKTPLSLNTPPGEVKLTLAGCEERNAQGMCSCAGFTQIVNEARIPACSL

SEQ ID NO: 16 - protein, 410 aa, synthetic construct
  REGION 1..410: note = APPA-Y14
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSMDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIQVSLVFQTLQQMRDKTPLSLNTPPGEVKLTLAGCEERNAQGMCSCAGFTQIVNEARIPACSL

SEQ ID NO: 17 - protein, 410 aa, synthetic construct
  REGION 1..410: note = APPA-Y15
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDIANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIQVSLVFQTLQQMRDKTPLSLNTPPGEVKLTLAGCEERNAQGMCSCAGFTQIVNEARIPACSL

SEQ ID NO: 18 - protein, 410 aa, synthetic construct
  REGION 1..410: note = APPA-Y16
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFMGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIQVSLVFQTLQQMRDKTPLSLNTPPGEVKLTLAGCEERNAQGMCSCAGFTQIVNEARIPACSL

SEQ ID NO: 19 - protein, 410 aa, synthetic construct
  REGION 1..410: note = APPA-Y17
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHLQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIQVSLVFQTLQQMRDKTPLSLNTPPGEVKLTLAGCEERNAQGMCSCAGFTQIVNEARIPACSL

SEQ ID NO: 20 - protein, 410 aa, synthetic construct
  REGION 1..410: note = APPA-Y18
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLPQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIQVSLVFQTLQQMRDKTPLSLNTPPGEVKLTLAGCEERNAQGMCSCAGFTQIVNEARIPACSL

SEQ ID NO: 21 - protein, 410 aa, synthetic construct
  REGION 1..410: note = APPA-Y19
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGALSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIQVSLVFQTLQQMRDKTPLSLNTPPGEVKLTLAGCEERNAQGMCSCAGFTQIVNEARIPACSL

SEQ ID NO: 22 - protein, 410 aa, synthetic construct
  REGION 1..410: note = APPA-Y20
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQYAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIQVSLVFQTLQQMRDKTPLSLNTPPGEVKLTLAGCEERNAQGMCSCAGFTQIVNEARIPACSL

SEQ ID NO: 23 - protein, 410 aa, synthetic construct
  REGION 1..410: note = APPA-Y21
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDWHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIQVSLVFQTLQQMRDKTPLSLNTPPGEVKLTLAGCEERNAQGMCSCAGFTQIVNEARIPACSL

SEQ ID NO: 24 - protein, 410 aa, synthetic construct
  REGION 1..410: note = APPA-Y22
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWYTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIQVSLVFQTLQQMRDKTPLSLNTPPGEVKLTLAGCEERNAQGMCSCAGFTQIVNEARIPACSL

SEQ ID NO: 25 - protein, 410 aa, synthetic construct
  REGION 1..410: note = APPA-Y23
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLFRTPEVARSRATPLLDLIKTALTPNGTQKQAYGVTLPTSVLFIAGHDTNLANLGGALELNWTLPGQPDNTPPGGELVFERWRRLSDNSCWIQVSLVFQTLQQMRDKTPLSLNTPPGEVKLTLAGCEERNAQGMCSCAGFTQIVNEARIPACSL

SEQ ID NO: 26 - protein, 410 aa, synthetic construct
  REGION 1..410: note = APPA-Y24
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARYRATPLLDLIKTALTPNGTQKQAYGVTLPTSVLFIAGHDTNLANLGGALELNWTLPGQPDNTPPGGELVFERWRRLSDNSCWIQVSLVFQTLQQMRDKTPLSLNTPPGEVKLTLAGCEERNAQGMCSCAGFTQIVNEARIPACSL

SEQ ID NO: 27 - protein, 410 aa, synthetic construct
  REGION 1..410: note = APPA-Y25
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPPSVLFIAGHDTNLANLGGALELNWTLPGQPDNTPPGGELVFERWRRLSDNSCWIQVSLVFQTLQQMRDKTPLSLNTPPGEVKLTLAGCEERNAQGMCSCAGFTQIVNEARIPACSL

SEQ ID NO: 28 - protein, 410 aa, synthetic construct
  REGION 1..410: note = APPA-Y26
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTKVLFIAGHDTNLANLGGALELNWTLPGQPDNTPPGGELVFERWRRLSDNSCWIQVSLVFQTLQQMRDKTPLSLNTPPGEVKLTLAGCEERNAQGMCSCAGFTQIVNEARIPACSL

SEQ ID NO: 29 - protein, 410 aa, synthetic construct
  REGION 1..410: note = APPA-Y27
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFLAGHDTNLANLGGALELNWTLPGQPDNTPPGGELVFERWRRLSDNSCWIQVSLVFQTLQQMRDKTPLSLNTPPGEVKLTLAGCEERNAQGMCSCAGFTQIVNEARIPACSL

SEQ ID NO: 30 - protein, 410 aa, synthetic construct
  REGION 1..410: note = APPA-Y28
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNIANLGGALELNWTLPGQPDNTPPGGELVFERWRRLSDNSCWIQVSLVFQTLQQMRDKTPLSLNTPPGEVKLTLAGCEERNAQGMCSCAGFTQIVNEARIPACSL

SEQ ID NO: 31 - protein, 410 aa, synthetic construct
  REGION 1..410: note = APPA-Y29
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSFWIQVSLVFQTLQQMRDKTPLSLNTPPGEVKLTLAGCEERNAQGMCSCAGFTQIVNEARIPACSL

SEQ ID NO: 32 - protein, 410 aa, synthetic construct
  REGION 1..410: note = APPA-Y30
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIKVSLVFQTLQQMRDKTPLSLNTPPGEVKLTLAGCEERNAQGMCSCAGFTQIVNEARIPACSL

SEQ ID NO: 33 - protein, 410 aa, synthetic construct
  REGION 1..410: note = APPA-Y31
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIQVSYVFQTLQQMRDKTPLSLNTPPGEVKLTLAGCEERNAQGMCSCAGFTQIVNEARIPACSL

SEQ ID NO: 34 - protein, 410 aa, synthetic construct
  REGION 1..410: note = APPA-Y32
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIQVSLVYQTLQQMRDKTPLSLNTPPGEVKLTLAGCEERNAQGMCSCAGFTQIVNEARIPACSL

SEQ ID NO: 35 - protein, 410 aa, synthetic construct
  REGION 1..410: note = APPA-Y33
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIQVSLVFQTLQQLRDKTPLSLNTPPGEVKLTLAGCEERNAQGMCSCAGFTQIVNEARIPACSL

SEQ ID NO: 36 - protein, 410 aa, synthetic construct
  REGION 1..410: note = APPA-Y34
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIQVSLVFQTLQQMRDATPLSLNTPPGEVKLTLAGCEERNAQGMCSCAGFTQIVNEARIPACSL

SEQ ID NO: 37 - protein, 410 aa, synthetic construct
  REGION 1..410: note = APPA-Y35
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIQVSLVFQTLQQMRDKTPLSLNCPPGEVKLTLAGCEERNAQGMCSCAGFTQIVNEARIPACSL

SEQ ID NO: 38 - protein, 410 aa, synthetic construct
  REGION 1..410: note = APPA-Y36
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIQVSLVFQTLQQMRDKTPLSLNTPPGRVKLTLAGCEERNAQGMCSCAGFTQIVNEARIPACSL

SEQ ID NO: 39 - protein, 410 aa, synthetic construct
  REGION 1..410: note = APPA-Y37
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIQVSLVFQTLQQMRDKTPLSLNTPPGEVKLTLAGCEERNAQGMCSCAGFIQIVNEARIPACSL

SEQ ID NO: 40 - protein, 410 aa, synthetic construct
  REGION 1..410: note = APPA-Y38
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPPSVLFIAGHDTNLANLGGALELNWTLPGQPDNTPPGGELVFERWRRLSDNSCWIKVSLVFQTLQQMRDKTPLSLNTPPGEVKLTLAGCEERNAQGMCSCAGFTQIVNEARIPACSL

SEQ ID NO: 41 - protein, 410 aa, synthetic construct
  REGION 1..410: note = APPA-Y39
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPPSVLFIAGHDTNLANLGGALELNWTLPGQPDNTPPGGELVFERWRRLSDNSCWIQVSLVFQTLQQMRDKTPLSLNTPPGRVKLTLAGCEERNAQGMCSCAGFTQIVNEARIPACSL

SEQ ID NO: 42 - protein, 410 aa, synthetic construct
  REGION 1..410: note = APPA-Y40
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIKVSLVFQTLQQMRDKTPLSLNTPPGRVKLTLAGCEERNAQGMCSCAGFTQIVNEARIPACSL

SEQ ID NO: 43 - protein, 410 aa, synthetic construct
  REGION 1..410: note = APPA-Y41
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPPSVLFIAGHDTNLANLGGALELNWTLPGQPDNTPPGGELVFERWRRLSDNSCWIKVSLVFQTLQQMRDKTPLSLNTPPGRVKLTLAGCEERNAQGMCSCAGFTQIVNEARIPACSL

SEQ ID NO: 44 - protein, 410 aa, synthetic construct
  REGION 1..410: note = APPA-Y25-1
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPASVLFIAGHDTNLANLGGALELNWTLPGQPDNTPPGGELVFERWRRLSDNSCWIQVSLVFQTLQQMRDKTPLSLNTPPGEVKLTLAGCEERNAQGMCSCAGFTQIVNEARIPACSL

SEQ ID NO: 45 - protein, 410 aa, synthetic construct
  REGION 1..410: note = APPA-Y25-2
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPCSVLFIAGHDTNLANLGGALELNWTLPGQPDNTPPGGELVFERWRRLSDNSCWIQVSLVFQTLQQMRDKTPLSLNTPPGEVKLTLAGCEERNAQGMCSCAGFTQIVNEARIPACSL

SEQ ID NO: 46 - protein, 410 aa, synthetic construct
  REGION 1..410: note = APPA-Y25-3
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPDSVLFIAGHDTNLANLGGALELNWTLPGQPDNTPPGGELVFERWRRLSDNSCWIQVSLVFQTLQQMRDKTPLSLNTPPGEVKLTLAGCEERNAQGMCSCAGFTQIVNEARIPACSL

SEQ ID NO: 47 - protein, 410 aa, synthetic construct
  REGION 1..410: note = APPA-Y25-4
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPESVLFIAGHDTNLANLGGALELNWTLPGQPDNTPPGGELVFERWRRLSDNSCWIQVSLVFQTLQQMRDKTPLSLNTPPGEVKLTLAGCEERNAQGMCSCAGFTQIVNEARIPACSL

SEQ ID NO: 48 - protein, 410 aa, synthetic construct
  REGION 1..410: note = APPA-Y25-5
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPFSVLFIAGHDTNLANLGGALELNWTLPGQPDNTPPGGELVFERWRRLSDNSCWIQVSLVFQTLQQMRDKTPLSLNTPPGEVKLTLAGCEERNAQGMCSCAGFTQIVNEARIPACSL

SEQ ID NO: 49 - protein, 410 aa, synthetic construct
  REGION 1..410: note = APPA-Y25-6
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPGSVLFIAGHDTNLANLGGALELNWTLPGQPDNTPPGGELVFERWRRLSDNSCWIQVSLVFQTLQQMRDKTPLSLNTPPGEVKLTLAGCEERNAQGMCSCAGFTQIVNEARIPACSL

SEQ ID NO: 50 - protein, 410 aa, synthetic construct
  REGION 1..410: note = APPA-Y25-7
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPHSVLFIAGHDTNLANLGGALELNWTLPGQPDNTPPGGELVFERWRRLSDNSCWIQVSLVFQTLQQMRDKTPLSLNTPPGEVKLTLAGCEERNAQGMCSCAGFTQIVNEARIPACSL

SEQ ID NO: 51 - protein, 410 aa, synthetic construct
  REGION 1..410: note = APPA-Y25-8
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPISVLFIAGHDTNLANLGGALELNWTLPGQPDNTPPGGELVFERWRRLSDNSCWIQVSLVFQTLQQMRDKTPLSLNTPPGEVKLTLAGCEERNAQGMCSCAGFTQIVNEARIPACSL

SEQ ID NO: 52 - protein, 410 aa, synthetic construct
  REGION 1..410: note = APPA-Y25-9
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPKSVLFIAGHDTNLANLGGALELNWTLPGQPDNTPPGGELVFERWRRLSDNSCWIQVSLVFQTLQQMRDKTPLSLNTPPGEVKLTLAGCEERNAQGMCSCAGFTQIVNEARIPACSL

SEQ ID NO: 53 - protein, 410 aa, synthetic construct
  REGION 1..410: note = APPA-Y25-10
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPLSVLFIAGHDTNLANLGGALELNWTLPGQPDNTPPGGELVFERWRRLSDNSCWIQVSLVFQTLQQMRDKTPLSLNTPPGEVKLTLAGCEERNAQGMCSCAGFTQIVNEARIPACSL

SEQ ID NO: 54 - protein, 410 aa, synthetic construct
  REGION 1..410: note = APPA-Y25-11
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPMSVLFIAGHDTNLANLGGALELNWTLPGQPDNTPPGGELVFERWRRLSDNSCWIQVSLVFQTLQQMRDKTPLSLNTPPGEVKLTLAGCEERNAQGMCSCAGFTQIVNEARIPACSL

SEQ ID NO: 55 - protein, 410 aa, synthetic construct
  REGION 1..410: note = APPA-Y25-12
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPNSVLFIAGHDTNLANLGGALELNWTLPGQPDNTPPGGELVFERWRRLSDNSCWIQVSLVFQTLQQMRDKTPLSLNTPPGEVKLTLAGCEERNAQGMCSCAGFTQIVNEARIPACSL

SEQ ID NO: 56 - protein, 410 aa, synthetic construct
  REGION 1..410: note = APPA-Y25-13
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPQSVLFIAGHDTNLANLGGALELNWTLPGQPDNTPPGGELVFERWRRLSDNSCWIQVSLVFQTLQQMRDKTPLSLNTPPGEVKLTLAGCEERNAQGMCSCAGFTQIVNEARIPACSL

SEQ ID NO: 57 - protein, 410 aa, synthetic construct
  REGION 1..410: note = APPA-Y25-14
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPRSVLFIAGHDTNLANLGGALELNWTLPGQPDNTPPGGELVFERWRRLSDNSCWIQVSLVFQTLQQMRDKTPLSLNTPPGEVKLTLAGCEERNAQGMCSCAGFTQIVNEARIPACSL

SEQ ID NO: 58 - protein, 410 aa, synthetic construct
  REGION 1..410: note = APPA-Y25-15
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPSSVLFIAGHDTNLANLGGALELNWTLPGQPDNTPPGGELVFERWRRLSDNSCWIQVSLVFQTLQQMRDKTPLSLNTPPGEVKLTLAGCEERNAQGMCSCAGFTQIVNEARIPACSL

SEQ ID NO: 59 - protein, 410 aa, synthetic construct
  REGION 1..410: note = APPA-Y25-16
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPVSVLFIAGHDTNLANLGGALELNWTLPGQPDNTPPGGELVFERWRRLSDNSCWIQVSLVFQTLQQMRDKTPLSLNTPPGEVKLTLAGCEERNAQGMCSCAGFTQIVNEARIPACSL

SEQ ID NO: 60 - protein, 410 aa, synthetic construct
  REGION 1..410: note = APPA-Y25-17
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPWSVLFIAGHDTNLANLGGALELNWTLPGQPDNTPPGGELVFERWRRLSDNSCWIQVSLVFQTLQQMRDKTPLSLNTPPGEVKLTLAGCEERNAQGMCSCAGFTQIVNEARIPACSL

SEQ ID NO: 61 - protein, 410 aa, synthetic construct
  REGION 1..410: note = APPA-Y25-18
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPYSVLFIAGHDTNLANLGGALELNWTLPGQPDNTPPGGELVFERWRRLSDNSCWIQVSLVFQTLQQMRDKTPLSLNTPPGEVKLTLAGCEERNAQGMCSCAGFTQIVNEARIPACSL

SEQ ID NO: 62 - protein, 410 aa, synthetic construct
  REGION 1..410: note = APPA-Y30-1
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIAVSLVFQTLQQMRDKTPLSLNTPPGEVKLTLAGCEERNAQGMCSCAGFTQIVNEARIPACSL

SEQ ID NO: 63 - protein, 410 aa, synthetic construct
  REGION 1..410: note = APPA-Y30-2
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWICVSLVFQTLQQMRDKTPLSLNTPPGEVKLTLAGCEERNAQGMCSCAGFTQIVNEARIPACSL

SEQ ID NO: 64 - protein, 410 aa, synthetic construct
  REGION 1..410: note = APPA-Y30-3
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIDVSLVFQTLQQMRDKTPLSLNTPPGEVKLTLAGCEERNAQGMCSCAGFTQIVNEARIPACSL

SEQ ID NO: 65 - protein, 410 aa, synthetic construct
  REGION 1..410: note = APPA-Y30-4
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIEVSLVFQTLQQMRDKTPLSLNTPPGEVKLTLAGCEERNAQGMCSCAGFTQIVNEARIPACSL

SEQ ID NO: 66 - protein, 410 aa, synthetic construct
  REGION 1..410: note = APPA-Y30-5
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIFVSLVFQTLQQMRDKTPLSLNTPPGEVKLTLAGCEERNAQGMCSCAGFTQIVNEARIPACSL

SEQ ID NO: 67 - protein, 410 aa, synthetic construct
  REGION 1..410: note = APPA-Y30-6
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIGVSLVFQTLQQMRDKTPLSLNTPPGEVKLTLAGCEERNAQGMCSCAGFTQIVNEARIPACSL

SEQ ID NO: 68 - protein, 410 aa, synthetic construct
  REGION 1..410: note = APPA-Y30-7
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIHVSLVFQTLQQMRDKTPLSLNTPPGEVKLTLAGCEERNAQGMCSCAGFTQIVNEARIPACSL

SEQ ID NO: 69 - protein, 410 aa, synthetic construct
  REGION 1..410: note = APPA-Y30-8
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIIVSLVFQTLQQMRDKTPLSLNTPPGEVKLTLAGCEERNAQGMCSCAGFTQIVNEARIPACSL

SEQ ID NO: 70 - protein, 410 aa, synthetic construct
  REGION 1..410: note = APPA-Y30-9
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWILVSLVFQTLQQMRDKTPLSLNTPPGEVKLTLAGCEERNAQGMCSCAGFTQIVNEARIPACSL

SEQ ID NO: 71 - protein, 410 aa, synthetic construct
  REGION 1..410: note = APPA-Y30-10
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIMVSLVFQTLQQMRDKTPLSLNTPPGEVKLTLAGCEERNAQGMCSCAGFTQIVNEARIPACSL

SEQ ID NO: 72 - protein, 410 aa, synthetic construct
  REGION 1..410: note = APPA-Y30-11
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWINVSLVFQTLQQMRDKTPLSLNTPPGEVKLTLAGCEERNAQGMCSCAGFTQIVNEARIPACSL

SEQ ID NO: 73 - protein, 410 aa, synthetic construct
  REGION 1..410: note = APPA-Y30-12
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIPVSLVFQTLQQMRDKTPLSLNTPPGEVKLTLAGCEERNAQGMCSCAGFTQIVNEARIPACSL

SEQ ID NO: 74 - protein, 410 aa, synthetic construct
  REGION 1..410: note = APPA-Y30-13
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIRVSLVFQTLQQMRDKTPLSLNTPPGEVKLTLAGCEERNAQGMCSCAGFTQIVNEARIPACSL

SEQ ID NO: 75 - protein, 410 aa, synthetic construct
  REGION 1..410: note = APPA-Y30-14
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWISVSLVFQTLQQMRDKTPLSLNTPPGEVKLTLAGCEERNAQGMCSCAGFTQIVNEARIPACSL

SEQ ID NO: 76 - protein, 410 aa, synthetic construct
  REGION 1..410: note = APPA-Y30-15
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWITVSLVFQTLQQMRDKTPLSLNTPPGEVKLTLAGCEERNAQGMCSCAGFTQIVNEARIPACSL

SEQ ID NO: 77 - protein, 410 aa, synthetic construct
  REGION 1..410: note = APPA-Y30-16
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIVVSLVFQTLQQMRDKTPLSLNTPPGEVKLTLAGCEERNAQGMCSCAGFTQIVNEARIPACSL

SEQ ID NO: 78 - protein, 410 aa, synthetic construct
  REGION 1..410: note = APPA-Y30-17
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIWVSLVFQTLQQMRDKTPLSLNTPPGEVKLTLAGCEERNAQGMCSCAGFTQIVNEARIPACSL

SEQ ID NO: 79 - protein, 410 aa, synthetic construct
  REGION 1..410: note = APPA-Y30-18
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIYVSLVFQTLQQMRDKTPLSLNTPPGEVKLTLAGCEERNAQGMCSCAGFTQIVNEARIPACSL

SEQ ID NO: 80 - protein, 410 aa, synthetic construct
  REGION 1..410: note = APPA-Y36-1
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIQVSLVFQTLQQMRDKTPLSLNTPPGAVKLTLAGCEERNAQGMCSCAGFTQIVNEARIPACSL

SEQ ID NO: 81 - protein, 410 aa, synthetic construct
  REGION 1..410: note = APPA-Y36-2
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIQVSLVFQTLQQMRDKTPLSLNTPPGCVKLTLAGCEERNAQGMCSCAGFTQIVNEARIPACSL

SEQ ID NO: 82 - protein, 410 aa, synthetic construct
  REGION 1..410: note = APPA-Y36-3
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIQVSLVFQTLQQMRDKTPLSLNTPPGDVKLTLAGCEERNAQGMCSCAGFTQIVNEARIPACSL

SEQ ID NO: 83 - protein, 410 aa, synthetic construct
  REGION 1..410: note = APPA-Y36-4
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIQVSLVFQTLQQMRDKTPLSLNTPPGFVKLTLAGCEERNAQGMCSCAGFTQIVNEARIPACSL

SEQ ID NO: 84 - protein, 410 aa, synthetic construct
  REGION 1..410: note = APPA-Y36-5
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIQVSLVFQTLQQMRDKTPLSLNTPPGGVKLTLAGCEERNAQGMCSCAGFTQIVNEARIPACSL

SEQ ID NO: 85 - protein, 410 aa, synthetic construct
  REGION 1..410: note = APPA-Y36-6
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIQVSLVFQTLQQMRDKTPLSLNTPPGHVKLTLAGCEERNAQGMCSCAGFTQIVNEARIPACSL

SEQ ID NO: 86 - protein, 410 aa, synthetic construct
  REGION 1..410: note = APPA-Y36-7
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIQVSLVFQTLQQMRDKTPLSLNTPPGIVKLTLAGCEERNAQGMCSCAGFTQIVNEARIPACSL

SEQ ID NO: 87 - protein, 410 aa, synthetic construct
  REGION 1..410: note = APPA-Y36-8
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIQVSLVFQTLQQMRDKTPLSLNTPPGKVKLTLAGCEERNAQGMCSCAGFTQIVNEARIPACSL

SEQ ID NO: 88 - protein, 410 aa, synthetic construct
  REGION 1..410: note = APPA-Y36-9
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIQVSLVFQTLQQMRDKTPLSLNTPPGLVKLTLAGCEERNAQGMCSCAGFTQIVNEARIPACSL

SEQ ID NO: 89 - protein, 410 aa, synthetic construct
  REGION 1..410: note = APPA-Y36-10
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIQVSLVFQTLQQMRDKTPLSLNTPPGMVKLTLAGCEERNAQGMCSCAGFTQIVNEARIPACSL

SEQ ID NO: 90 - protein, 410 aa, synthetic construct
  REGION 1..410: note = APPA-Y36-11
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIQVSLVFQTLQQMRDKTPLSLNTPPGNVKLTLAGCEERNAQGMCSCAGFTQIVNEARIPACSL

SEQ ID NO: 91 - protein, 410 aa, synthetic construct
  REGION 1..410: note = APPA-Y36-12
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIQVSLVFQTLQQMRDKTPLSLNTPPGPVKLTLAGCEERNAQGMCSCAGFTQIVNEARIPACSL

SEQ ID NO: 92 - protein, 410 aa, synthetic construct
  REGION 1..410: note = APPA-Y36-13
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIQVSLVFQTLQQMRDKTPLSLNTPPGQVKLTLAGCEERNAQGMCSCAGFTQIVNEARIPACSL

SEQ ID NO: 93 - protein, 410 aa, synthetic construct
  REGION 1..410: note = APPA-Y36-14
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIQVSLVFQTLQQMRDKTPLSLNTPPGSVKLTLAGCEERNAQGMCSCAGFTQIVNEARIPACSL

SEQ ID NO: 94 - protein, 410 aa, synthetic construct
  REGION 1..410: note = APPA-Y36-15
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIQVSLVFQTLQQMRDKTPLSLNTPPGTVKLTLAGCEERNAQGMCSCAGFTQIVNEARIPACSL

SEQ ID NO: 95 - protein, 410 aa, synthetic construct
  REGION 1..410: note = APPA-Y36-16
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIQVSLVFQTLQQMRDKTPLSLNTPPGVVKLTLAGCEERNAQGMCSCAGFTQIVNEARIPACSL

SEQ ID NO: 96 - protein, 410 aa, synthetic construct
  REGION 1..410: note = APPA-Y36-17
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIQVSLVFQTLQQMRDKTPLSLNTPPGWVKLTLAGCEERNAQGMCSCAGFTQIVNEARIPACSL

SEQ ID NO: 97 - protein, 410 aa, synthetic construct
  REGION 1..410: note = APPA-Y36-18
QSEPELKLESVVIVSRHGVRAPTKATQLMQDVTPDAWPTWPVKLGELTPRGGELIAYLGHYWRQRLVADELLPNQTCPQSGQVAIIADVDERTRKTGEAFAAGLAPDCAITVHTQADTSSPDPLFNPLKTGVCQLDNANVTDAILERAGGSIADFTGHYQTAFRELERVLNFSQSNLCLKREKQDESCSLTQALPSELKVSADNVSLTGAVSLASMLTEIFLLQQAQGMPEPGWGRITDSHQWNTLLSLHNAQFDLLQRTPEVARSRATPLLDLIKTALTPNGTQKQAYGVTLPTSVLFIAGHDTNLANLGGALELNWTLPGQPDNTPPGGELVFERWRRLSDNSCWIQVSLVFQTLQQMRDKTPLSLNTPPGYVKLTLAGCEERNAQGMCSCAGFTQIVNEARIPACSL

SEQ ID NO: 98 - protein, 410 aa, synthetic construct
  REGION 1..410: note = APPA-An1
QSEEELKLESVVIVSRHGVRAPTKFTQLMQDVTPYAWPTWPVKLGELTPRGGELIAYLGHYWRQRLVADELLPNQTCPQPGQVAIIADVDERTRKTGEAFAAGLAPGCAITVHHQADTSSPDPLFNPLKTGVCQLDVARVTRAILERAGGSIADFTNHYQPAFRELERVLNFSQSPLCKNREKQNEPCSLTQALPSELKVSADNVSLTGAWSLASMLTEIFLLQQAQGMPEPGWGRITDSHQWNTLLSLHNAYFDLLQRTPEVARSAATPLLDLIKTALTPNGTQKSAYGVTLPTSVLFIAGHDTNLANLGGALELNWTLPGQPDNYPPGGELVFERWRRLSDNSCWIQVSLVFQTLQQMRDKTPLSLNTPPGEVKLTLPGCEERNAQGMCSCAGFTQIVNEARIPACSL

SEQ ID NO: 99 - protein, 410 aa, synthetic construct
  REGION 1..410: note = APPA-An2
QSEEELKLESVVIVSRHGVRAPTKFTQLMQDVTPYAWPTWPVKLGELTPRGGELIAYLGHYWRQRLVADELLPNQTCPQPGQVAIIADVDERTRKTGEAFAAGLAPGCAITVHHQADTSSPDPLFNPLKTGVCQLDVARVTRAILERAGGSIADFTNHYQPAFRELERVLNFSQSPLCKNREKQNEPCSLTQALPSELKVSADNVSLTGAWSLASMLTEIFLLQQAQGMPEPGWGRITDSHQWNTLLSLHNAYFDLLQRTPEVARSAATPLLDLIKTALTPNGTQKSAYGVTLPTSVLFIAGHDTNLANLGGALELNWTLPGQPDNYPPGGELVFERWRRLSDNSCWIKVSLVFQTLQQMRDKTPLSLNTPPGEVKLTLPGCEERNAQGMCSCAGFTQIVNEARIPACSL

SEQ ID NO: 100 - protein, 410 aa, synthetic construct
  REGION 1..410: note = APPA-An3
QSEEELKLESVVIVSRHGVRAPTKFTQLMQDVTPYAWPTWPVKLGELTPRGGELIAYLGHYWRQRLVADELLPNQTCPQPGQVAIIADVDERTRKTGEAFAAGLAPGCAITVHHQADTSSPDPLFNPLKTGVCQLDVARVTRAILERAGGSIADFTNHYQPAFRELERVLNFSQSPLCKNREKQNEPCSLTQALPSELKVSADNVSLTGAWSLASMLTEIFLLQQAQGMPEPGWGRITDSHQWNTLLSLHNAYFDLLQRTPEVARSAATPLLDLIKTALTPNGTQKSAYGVTLPTSVLFIAGHDTNLANLGGALELNWTLPGQPDNYPPGGELVFERWRRLSDNSCWIQVSLVFQTLQQMRDKTPLSLNTPPGRVKLTLPGCEERNAQGMCSCAGFTQIVNEARIPACSL

SEQ ID NO: 101 - protein, 410 aa, synthetic construct
  REGION 1..410: note = APPA-An4
QSEEELKLESVVIVSRHGVRAPTKFTQLMQDVTPYAWPTWPVKLGELTPRGGELIAYLGHYWRQRLVADELLPNQTCPQPGQVAIIADVDERTRKTGEAFAAGLAPGCAITVHHQADTSSPDPLFNPLKTGVCQLDVARVTRAILERAGGSIADFTNHYQPAFRELERVLNFSQSPLCKNREKQNEPCSLTQALPSELKVSADNVSLTGAWSLASMLTEIFLLQQAQGMPEPGWGRITDSHQWNTLLSLHNAYFDLLQRTPEVARSAATPLLDLIKTALTPNGTQKSAYGVTLPTSVLFIAGHDTNLANLGGALELNWTLPGQPDNYPPGGELVFERWRRLSDNSCWIKVSLVFQTLQQMRDKTPLSLNTPPGRVKLTLPGCEERNAQGMCSCAGFTQIVNEARIPACSL

SEQ ID NO: 102 - protein, 410 aa, synthetic construct
  REGION 1..410: note = APPA-An5
QSEEELKLESVVIVSRHGVRAPTKFTQLMQDVTPYAWPTWPVKLGELTPRGGELIAYLGHYWRQRLVADELLPNQTCPQPGQVAIIADVDERTRKTGEAFAAGLAPDCAITVHHQNDTSSPDPLFNPLKTGVCQLDVARVTRAILERAGGSIADFTNHYQPAFRELERVLNFSQSPLCKNREKQNEPCSLTQALPSELKVSADNVSLTGAWSLASMLTEIFLLQQAQGMPEPGWGRITDSHQWNTLLSLHNAQFDLLQRTPEVARSAATPLLDLIKTALTPNGTQKQAYGVTLPTSVLFIAGHDTNLANLGGALELNWTLPGQPDNYPPGGELVFERWRRLSDNSCWIQVSLVFQTLQQMRDKTPLSLNTPPGEVKLTLPGCEERNAQGMCSCAGFTQIVNEARIPACSL

SEQ ID NO: 103 - protein, 410 aa, synthetic construct
  REGION 1..410: note = APPA-An6
QSEEELKLESVVIVSRHGVRAPTKFTQLMQDVTPYAWPTWPVKLGELTPRGGELIAYLGHYWRQRLVADELLPNQTCPQPGQVAIIADVDERTRKTGEAFAAGLAPDCAITVHHQNDTSSPDPLFNPLKTGVCQLDVARVTRAILERAGGSIADFTNHYQPAFRELERVLNFSQSPLCKNREKQNEPCSLTQALPSELKVSADNVSLTGAWSLASMLTEIFLLQQAQGMPEPGWGRITDSHQWNTLLSLHNAQFDLLQRTPEVARSAATPLLDLIKTALTPNGTQKQAYGVTLPPSVLFIAGHDTNLANLGGALELNWTLPGQPDNYPPGGELVFERWRRLSDNSCWIQVSLVFQTLQQMRDKTPLSLNTPPGEVKLTLPGCEERNAQGMCSCAGFTQIVNEARIPACSL

SEQ ID NO: 104 - protein, 410 aa, synthetic construct
  REGION 1..410: note = APPA-An7
QSEEELKLESVVIVSRHGVRAPTKFTQLMQDVTPYAWPTWPVKLGELTPRGGELIAYLGHYWRQRLVADELLPNQTCPQPGQVAIIADVDERTRKTGEAFAAGLAPDCAITVHHQNDTSSPDPLFNPLKTGVCQLDVARVTRAILERAGGSIADFTNHYQPAFRELERVLNFSQSPLCKNREKQNEPCSLTQALPSELKVSADNVSLTGAWSLASMLTEIFLLQQAQGMPEPGWGRITDSHQWNTLLSLHNAQFDLLQRTPEVARSAATPLLDLIKTALTPNGTQKQAYGVTLPTSVLFIAGHDTNLANLGGALELNWTLPGQPDNYPPGGELVFERWRRLSDNSCWIKVSLVFQTLQQMRDKTPLSLNTPPGEVKLTLPGCEERNAQGMCSCAGFTQIVNEARIPACSL

SEQ ID NO: 105 - protein, 410 aa, synthetic construct
  REGION 1..410: note = APPA-An8
QSEEELKLESVVIVSRHGVRAPTKFTQLMQDVTPYAWPTWPVKLGELTPRGGELIAYLGHYWRQRLVADELLPNQTCPQPGQVAIIADVDERTRKTGEAFAAGLAPDCAITVHHQNDTSSPDPLFNPLKTGVCQLDVARVTRAILERAGGSIADFTNHYQPAFRELERVLNFSQSPLCKNREKQNEPCSLTQALPSELKVSADNVSLTGAWSLASMLTEIFLLQQAQGMPEPGWGRITDSHQWNTLLSLHNAQFDLLQRTPEVARSAATPLLDLIKTALTPNGTQKQAYGVTLPTSVLFIAGHDTNLANLGGALELNWTLPGQPDNYPPGGELVFERWRRLSDNSCWIQVSLVFQTLQQMRDKTPLSLNTPPGRVKLTLPGCEERNAQGMCSCAGFTQIVNEARIPACSL

SEQ ID NO: 106 - protein, 410 aa, synthetic construct
  REGION 1..410: note = APPA-An9
QSEEELKLESVVIVSRHGVRAPTKFTQLMQDVTPYAWPTWPVKLGELTPRGGELIAYLGHYWRQRLVADELLPNQTCPQPGQVAIIADVDERTRKTGEAFAAGLAPDCAITVHHQNDTSSPDPLFNPLKTGVCQLDVARVTRAILERAGGSIADFTNHYQPAFRELERVLNFSQSPLCKNREKQNEPCSLTQALPSELKVSADNVSLTGAWSLASMLTEIFLLQQAQGMPEPGWGRITDSHQWNTLLSLHNAQFDLLQRTPEVARSAATPLLDLIKTALTPNGTQKQAYGVTLPTSVLFIAGHDTNLANLGGALELNWTLPGQPDNYPPGGELVFERWRRLSDNSCWIKVSLVFQTLQQMRDKTPLSLNTPPGRVKLTLPGCEERNAQGMCSCAGFTQIVNEARIPACSL

SEQ ID NO: 107 - protein, 410 aa, synthetic construct
  REGION 1..410: note = APPA-An10
QSEEELKLESVVIVSRHGVRAPTKFTQLMQDVTPDAWPTWPVKLGELTPRGGELIAYLGHYWRQRLVADELLPNQTCPQPGQVAIIADVDERTRKTGEAFAAGLAPGCAITVHHQNDTSSPDPLFNPLKTGVCQLDVARVTRAILERAGGSIADFTNHYQPAFRELERVLNFSQSPLCKNREKQNEPCSLTQALPSELKVSADNVSLTGAWSLASMLTEIFLLQQAQGMPEPGWGRITDSHQWNTLLSLHNAYFDLLQRTPEVARSRATPLLDLIKTALTPNGTQKSAYGVTLPTSVLFIAGHDTNLANLGGALELNWTLPGQPDNTPPGGELVFERWRRLSDNSCWIQVSLVFQTLQQMRDKTPLSLNTPPGEVKLTLPGCEERNAQGMCSCAGFTQIVNEARIPACSL

SEQ ID NO: 108 - protein, 410 aa, synthetic construct
  REGION 1..410: note = APPA-An11
QSEEELKLESVVIVSRHGVRAPTKFTQLMQDVTPDAWPTWPVKLGELTPRGGELIAYLGHYWRQRLVADELLPNQTCPQPGQVAIIADVDERTRKTGEAFAAGLAPGCAITVHHQNDTSSPDPLFNPLKTGVCQLDVARVTRAILERAGGSIADFTNHYQPAFRELERVLNFSQSPLCKNREKQNEPCSLTQALPSELKVSADNVSLTGAWSLASMLTEIFLLQQAQGMPEPGWGRITDSHQWNTLLSLHNAYFDLLQRTPEVARSRATPLLDLIKTALTPNGTQKSAYGVTLPTSVLFIAGHDTNLANLGGALELNWTLPGQPDNTPPGGELVFERWRRLSDNSCWIQVSLVFQTLQQMRDKTPLSLNTPPGRVKLTLPGCEERNAQGMCSCAGFTQIVNEARIPACSL

SEQ ID NO: 109 - protein, 410 aa, synthetic construct
  REGION 1..410: note = APPA-An12
QSEEELKLESVVIVSRHGVRAPTKFTQLMQDVTPDAWPTWPVKLGELTPRGGELIAYLGHYWRQRLVADELLPNQTCPQPGQVAIIADVDERTRKTGEAFAAGLAPGCAITVHHQNDTSSPDPLFNPLKTGVCQLDVARVTRAILERAGGSIADFTNHYQPAFRELERVLNFSQSPLCKNREKQNEPCSLTQALPSELKVSADNVSLTGAWSLASMLTEIFLLQQAQGMPEPGWGRITDSHQWNTLLSLHNAYFDLLQRTPEVARSRATPLLDLIKTALTPNGTQKSAYGVTLPPSVLFIAGHDTNLANLGGALELNWTLPGQPDNTPPGGELVFERWRRLSDNSCWIKVSLVFQTLQQMRDKTPLSLNTPPGEVKLTLPGCEERNAQGMCSCAGFTQIVNEARIPACSL

SEQ ID NO: 110 - protein, 410 aa, synthetic construct
  REGION 1..410: note = APPA-An13
QSEEELKLESVVIVSRHGVRAPTKFTQLMQDVTPDAWPTWPVKLGELTPRGGELIAYLGHYWRQRLVADELLPNQTCPQPGQVAIIADVDERTRKTGEAFAAGLAPGCAITVHHQNDTSSPDPLFNPLKTGVCQLDVARVTRAILERAGGSIADFTNHYQPAFRELERVLNFSQSPLCKNREKQNEPCSLTQALPSELKVSADNVSLTGAWSLASMLTEIFLLQQAQGMPEPGWGRITDSHQWNTLLSLHNAYFDLLQRTPEVARSRATPLLDLIKTALTPNGTQKSAYGVTLPPSVLFIAGHDTNLANLGGALELNWTLPGQPDNTPPGGELVFERWRRLSDNSCWIQVSLVFQTLQQMRDKTPLSLNTPPGRVKLTLPGCEERNAQGMCSCAGFTQIVNEARIPACSL

SEQ ID NO: 111 - protein, 410 aa, synthetic construct
  REGION 1..410: note = APPA-An14
QSEEELKLESVVIVSRHGVRAPTKFTQLMQDVTPDAWPTWPVKLGELTPRGGELIAYLGHYWRQRLVADELLPNQTCPQPGQVAIIADVDERTRKTGEAFAAGLAPGCAITVHHQNDTSSPDPLFNPLKTGVCQLDVARVTRAILERAGGSIADFTNHYQPAFRELERVLNFSQSPLCKNREKQNEPCSLTQALPSELKVSADNVSLTGAWSLASMLTEIFLLQQAQGMPEPGWGRITDSHQWNTLLSLHNAYFDLLQRTPEVARSRATPLLDLIKTALTPNGTQKSAYGVTLPPSVLFIAGHDTNLANLGGALELNWTLPGQPDNTPPGGELVFERWRRLSDNSCWIKVSLVFQTLQQMRDKTPLSLNTPPGRVKLTLPGCEERNAQGMCSCAGFTQIVNEARIPACSL

SEQ ID NO: 112 - protein, 410 aa, synthetic construct
  REGION 1..410: note = APPA-An15
QSEEELKLESVVIVSRHGVRAPTKFTQLMQDVTPYAWPTWPVKLGELTPRGGELIAYLGHYWRQRLVADELLPNQTCPQPGQVAIIADVDERTRKTGEAFAAGLAPGCAITVHHQADTSSPDPLFNPLKTGVCQLDVARVTRAILERAGGSIADFTNHYQPAFRELERVLNFSQSPLCLNREKQNEPCSLTQALPSELKVSADNVSLTGAWSLASMLTEIFLLQQAEGMPEPGWGRITDSHQWNTLLSLHNAYFDLLQRTPEVARSRATPLLDLIKTALTPNGTQKQAYGVTLPTSVLFIAGHDTNLANLGGALELNWTLPGQPDNYPPGGELVFERWRRLSDNSCWIQVSLVFQTLQQMRDKTPLSLNTPPGEVKLTLPGCEERNAQGMCSCAGFTQIVNEARIPACSL

SEQ ID NO: 113 - protein, 410 aa, synthetic construct
  REGION 1..410: note = APPA-An16
QSEEELKLESVVIVSRHGVRAPTKFTQLMQDVTPYAWPTWPVKLGELTPRGGELIAYLGHYWRQRLVADELLPNQTCPQPGQVAIIADVDERTRKTGEAFAAGLAPGCAITVHHQADTSSPDPLFNPLKTGVCQLDVARVTRAILERAGGSIADFTNHYQPAFRELERVLNFSQSPLCLNREKQNEPCSLTQALPSELKVSADNVSLTGAWSLASMLTEIFLLQQAEGMPEPGWGRITDSHQWNTLLSLHNAYFDLLQRTPEVARSRATPLLDLIKTALTPNGTQKQAYGVTLPPSVLFIAGHDTNLANLGGALELNWTLPGQPDNYPPGGELVFERWRRLSDNSCWIQVSLVFQTLQQMRDKTPLSLNTPPGEVKLTLPGCEERNAQGMCSCAGFTQIVNEARIPACSL

SEQ ID NO: 114 - protein, 410 aa, synthetic construct
  REGION 1..410: note = APPA-An17
QSEEELKLESVVIVSRHGVRAPTKFTQLMQDVTPYAWPTWPVKLGELTPRGGELIAYLGHYWRQRLVADELLPNQTCPQPGQVAIIADVDERTRKTGEAFAAGLAPGCAITVHHQADTSSPDPLFNPLKTGVCQLDVARVTRAILERAGGSIADFTNHYQPAFRELERVLNFSQSPLCLNREKQNEPCSLTQALPSELKVSADNVSLTGAWSLASMLTEIFLLQQAEGMPEPGWGRITDSHQWNTLLSLHNAYFDLLQRTPEVARSRATPLLDLIKTALTPNGTQKQAYGVTLPTSVLFIAGHDTNLANLGGALELNWTLPGQPDNYPPGGELVFERWRRLSDNSCWIQVSLVFQTLQQMRDKTPLSLNTPPGRVKLTLPGCEERNAQGMCSCAGFTQIVNEARIPACSL

SEQ ID NO: 115 - protein, 410 aa, synthetic construct
  REGION 1..410: note = APPA-An18
QSEEELKLESVVIVSRHGVRAPTKFTQLMQDVTPYAWPTWPVKLGELTPRGGELIAYLGHYWRQRLVADELLPNQTCPQPGQVAIIADVDERTRKTGEAFAAGLAPGCAITVHHQADTSSPDPLFNPLKTGVCQLDVARVRRAILERAGGPIADFTNHYQPAFRELERVLNFSQSNLCKKREKQNEPCSLTQALPSELKVSADNVSLTGAWSLASMLTEIFLLQQAQGMPEPGWGRITDSHQWNTLLSLHNAYFDLLQRTPEVARSAATPLLDLIKTALTPNGTQKSAYGVTLPTSVLFIAGHDTNLANLGGALELNWTLPGQPDNYPPGGELVFERWRRLSDNSCWIQVSLVFQTLQQMRDKTPLSLNTPPGEVKLTLPGCEERNAQGMCSCAGFTQIVNEARIPACSL

SEQ ID NO: 116 - protein, 410 aa, synthetic construct
  REGION 1..410: note = APPA-An19
QSEEELKLESVVIVSRHGVRAPTKFTQLMQDVTPYAWPTWPVKLGELTPRGGELIAYLGHYWRQRLVADELLPNQTCPQPGQVAIIADVDERTRKTGEAFAAGLAPGCAITVHHQADTSSPDPLFNPLKTGVCQLDVARVRRAILERAGGPIADFTNHYQPAFRELERVLNFSQSNLCKKREKQNEPCSLTQALPSELKVSADNVSLTGAWSLASMLTEIFLLQQAQGMPEPGWGRITDSHQWNTLLSLHNAYFDLLQRTPEVARSAATPLLDLIKTALTPNGTQKSAYGVTLPTSVLFIAGHDTNLANLGGALELNWTLPGQPDNYPPGGELVFERWRRLSDNSCWIKVSLVFQTLQQMRDKTPLSLNTPPGEVKLTLPGCEERNAQGMCSCAGFTQIVNEARIPACSL

SEQ ID NO: 117 - protein, 410 aa, synthetic construct
  REGION 1..410: note = APPA-An20
QSEEELKLESVVIVSRHGVRAPTKFTQLMQDVTPYAWPTWPVKLGELTPRGGELIAYLGHYWRQRLVADELLPNQTCPQPGQVAIIADVDERTRKTGEAFAAGLAPGCAITVHHQADTSSPDPLFNPLKTGVCQLDVARVRRAILERAGGPIADFTNHYQPAFRELERVLNFSQSNLCKKREKQNEPCSLTQALPSELKVSADNVSLTGAWSLASMLTEIFLLQQAQGMPEPGWGRITDSHQWNTLLSLHNAYFDLLQRTPEVARSAATPLLDLIKTALTPNGTQKSAYGVTLPTSVLFIAGHDTNLANLGGALELNWTLPGQPDNYPPGGELVFERWRRLSDNSCWIKVSLVFQTLQQMRDKTPLSLNTPPGRVKLTLPGCEERNAQGMCSCAGFTQIVNEARIPACSL

SEQ ID NO: 118 - protein, 410 aa, synthetic construct
  REGION 1..410: note = APPA-An21
QSEEELKLESVVIVSRHGVRAPTKFTQLMQDVTPYAWPTWPVKLGELTPRGGELIAYLGHYWRQRLVADELLPNQTCPQPGQVAIIADVDERTRKTGEAFAAGLAPGCAITVHHQADTSSPDPLFNPLKTGVCQLDVARVRRAILERAGGPIADFTNHYQPAFRELERVLNFSQSNLCKKREKQNEPCSLTQALPSELKVSADNVSLTGAWSLASMLTEIFLLQQAQGMPEPGWGRITDSHQWNTLLSLHNAYFDLLQRTPEVARSAATPLLDLIKTALTPNGTQKSAYGVTLPPSVLFIAGHDTNLANLGGALELNWTLPGQPDNYPPGGELVFERWRRLSDNSCWIKVSLVFQTLQQMRDKTPLSLNTPPGRVKLTLPGCEERNAQGMCSCAGFTQIVNEARIPACSL